SEQUENCE LISTING (122 sequences)

SEQ ID NO: 1 - DNA, 439 bp, Homo Sapien
  CDS (45)...(305)
ttgagctgcaggttgaatccggggtgcctttaggattcagcaccatggcggaagacatggagaccaaaatcaagaactacaagaccgccccttttgacagccgcttccccaaccagaaccagactagaaactgctggcagaactacctggacttccaccgctgtcagaaggcaatgaccgctaaaggaggcgatatctctgtgtgcgaatggtaccagcgtgtgtaccagtccctctgccccacatcctgggtcacagactgggatgagcaacgggctgaaggcacgtttcccgggaagatctgaactggctgcatctccctttcctctgtcctccatccttctcccaggatggtgaagggggacctggtacccagtgatccccaccccaggatcctaaatcatgacttacctgctaataaaaactcattggaaaagtg

SEQ ID NO: 2 - protein, 86 aa, Homo Sapien
MAEDMETKIKNYKTAPFDSRFPNQNQTRNCWQNYLDFHRCQKAMTAKGGDISVCEWYQRVYQSLCPTSWVTDWDEQRAEGTFPGKI

SEQ ID NO: 3 - DNA, 18 bp, Artificial Sequence
  PCR Primer
aggattcagcaccatggc

SEQ ID NO: 4 - DNA, 43 bp, Artificial Sequence
  PCR Primer
agcggataacaatttcacacaggtagtctggttctggttgggg

SEQ ID NO: 5 - DNA, 18 bp, Artificial Sequence
  MassExtend primer
aatcaagaactacaagac

SEQ ID NO: 6 - DNA, 2921 bp, Homo Sapien
  CDS (103)...(1848)
cagcgagcgccgtcgtctgcccgggcccgcccatcggggtccccaaccccatccggaccccgccgcccgagcgcgcggccccggaagcacccgcctcccggcatggtgctcaaggccttcttccccacgtgctgcgtctcggcggacagcgggctgctggtgggacggtgggtgccggagcagagcagcgccgtggtcctggcggtcctgcactttcccttcatccccatccaggtcaagcagctcctggcccaggtgcggcaggccagccaggtgggcgtggccgtgctgggcacctggtgccactgccggcaggagcccgaggagagcctgggccgcttcctggagagcctgggtgctgtcttcccccatgagccctggctgcggctgtgccgggagagaggcggcacgttctggagctgcgaggccacccaccggcaagcgcccactgcccccggtgcccctggtgaggaccaggtcatgctcatcttctatgaccagcgccaggtgttgctgtcacagctacacctgcccaccgtcctgcccgaccgccaggctggagccaccactgccagcacggggggcctggctgccgtcttcgacacggtagcacgcagtgaggtgctcttccgcagtgaccgctttgatgagggccccgtgcggctgagccactggcagtcggagggcgtggaggccagcatcctcgcggagctggccaggcgagcctcgggacccatttgtctgctgttggccagcctgctgtcgctggtctcagctgtcagtgcctgccgagtgttcaagctctggcccctgtccttcctcgggagcaaactctccacgtgcgaacagctccggcaccggctggagcacctcacgctaatcttcagtacacggaaggcggagaaccctgcccagctgatgaggaaggccaacacggtggcctctgtgctgctggacgtggccctgggcctcatgctgctgtcctggctccacgggagaagccgcatcgggcatctggccgacgccctcgttcctgtggctgaccacgtggccgaggagctccagcatctgctgcagtggctgatgggtgctcccgccgggctcaagatgaaccgtgcactggaccaggtgctgggccgcttcttcctctaccacatccacctgtggatcagctacatccacctcatgtcccccttcgtggagcacatcctttggcacgtgggcctctcggcctgcctgggcctgacggtggccctgtccctcctctcggacattatcgccctcctcaccttccacatctactgcttttacgtctatggagccaggctgtactgcctgaagatccatggcctgtcctcactgtggcgtctgttccgggggaagaagtggaacgttctgcgccagcgcgtggactcctgttcctatgacctggaccagctgttcatcgggactctgctcttcaccatcctgctcttcctcctgcctaccacagccctgtactacctggtgttcaccctgctccggctcctggtggtcgccgtgcagggcctgatccatctgctggtggacctcatcaactccctgccgctgtactcactgggtcttcggctctgccggccctacaggctggcggctggcgtgaagttccgtgtcctccggcacgaggccagcaggcccctccgcctcctgatgcagataaacccactgccctacagccgcgtggtgcacacctaccgcctccccagctgtggctgccaccccaagcactcctggggcgccctgtgccgcaagctgttccttggggagctcatctacccctggaggcagagaggggacaagcaggactgagggaactgctggctcgcctggcaccaccacacggccacagccagccatctgctctgccagggtggcaccagctcagctggcgcatgtcccgtgctttgtggacgctgctgtgtgctcctgaacacggcaggccctgctatcacaccttgggcttggaggtcattgggagtgagcagatgtgggggtggccagccaggctggccgcactccatcactggcactgcctgccttgggacccgcttcccacctgctgcggtcaccatggtggcgagcacagcaaccccaggtgtccagagcactgccccatgcccaccctgcatacccaggtccagagggtccgtccaccacagcagccccaggtggagggctggtctccctgggggctccccagtggctctgccctggctgtgggggtggagggaccttgccaggatgaaccctccagtcccaggcaccctctagctccctcagccgaacagcaccctgcatctgggggattgaagcagtcgctgacccccgtccccagcgggcccgggccctcactccctgaaccacacggggtttatttgcggatgttccctggagaggtcgctttgtgaagaaaccatcagcaggctgtgagcatcgccaggctgctgtgggggcgggagcagcctcagtgtcaagggcctgcccactgacccagccgtacctattcgtccacggtgccccgtagcagcaggtcctgcggccaaatctgtctcccttcatgggcctcccagggaaggaggaagccctgctgtgcagacacctctgtggccccccaggggtgtgagcggcctggggagggggccgtggcactgaggccgaaagtgcctgccagacggcacggtctgggtgcgggtgttccctgtgagcccgagtccgcttcaggaggggagcctgcaggtgccggctggtgaggggatgacgcgctgtgggtgggaggaggcagcgcccatctcagcagcaccaggactgcctgggactccctggcaacccagcaccggggaagccgtcagctgctgtgacaataaaacctgccccgtgtctgg

SEQ ID NO: 7 - protein, 581 aa, Homo Sapien [sequence omitted]

SEQ ID NO: 8 - DNA, 18 bp, Artificial Sequence
  PCR primer
agcagggcttcctccttc

SEQ ID NO: 9 - DNA, 43 bp, Artificial Sequence
  PCR primer
agcggataacaatttcacacaggtgacccagccgtacctattc

SEQ ID NO: 10 - DNA, 18 bp, Artificial Sequence
  MassExtend primer
aagggagacagatttggc

SEQ ID NO: 11 - DNA, 1790 bp, Homo sapien
  CDS (131)...(1612): Nucleotide sequence encoding Cholesterol ester transfer protein (CETP)
gtgaatctctggggccaggaagaccctgctgcccggaagagcctcatgttccgtgggggctgggcggacatacatatacgggctccaggctgaacggctcgggccacttacacaccactgcctgataaccatgctggctgccacagtcctgaccctggccctgctgggcaatgcccatgcctgctccaaaggcacctcgcacgaggcaggcatcgtgtgccgcatcaccaagcctgccctcctggtgttgaaccacgagactgccaaggtgatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctagaagtctccaaggaggtcgggatggggcttgtagcagaaggcaagcaccaggctcacagctggaaccctggtgtctcctccagcgtggtggaagttgggttaggagtacggagatggagattggctcccaactcctccctatcctaaaggcccactggcattaaagtgctgtatccaag

SEQ ID NO: 12 - protein, 493 aa, Homo sapien
MLAATVLTLALLGNAHACSKGTSHEAGIVCRITKPALLVLNHETAKVIQTAFQRASYPDITGEKAMMLLGQVKYGLHNIQISHLSIASSQVELVEAKSIDVSIQNVSVVFKGTLKYGYTTAWWLGIDQSIDFEIDSAIDLQINTQLTCDSGRVRTDAPDCYLSFHKLLLHLQGEREPGWIKQLFTNFISFTLKLVLKGQICKEINVISNIMADFVQTRAASILSDGDIGVDISLTGDPVITASYLESHHKGHFIYKNVSEDLPLPTFSPTLLGDSRMLYFWFSERVFHSLAKVAFQDGRLMLSLMGDEFKAVLETWGFNTNQEIFQEVVGGFPSQAQVTVHCLKMPKISCQNKGVVVNSSVMVKFLFPRPDQQHSVAYTFEEDIVTTVQASYSKKKLFLSLLDFQITPKTVSNLTESSSESIQSFLQSMITAVGIPEVMSRLEVVFTALMNSKGVSLFDIINPEIITRDGFLLLQMDFGFPEHLLVDFLQSLS

SEQ ID NO: 13 - DNA, 3549 bp, Homo sapien
  CDS (175)...(1602): Nucleotide sequence encoding lipoprotein lipase (LPL)
cccctcttcctcctcctcaagggaaagctgcccacttctagctgccctgccatcccctttaaagggcgacttgctcagcgccaaaccgcggctccagccctctccagcctccggctcagccggctcatcagtcggtccgcgccttgcagctcctccagagggacgcgccccgagatggagagcaaagccctgctcgtgctgactctggccgtgtggctccagagtctgaccgcctcccgcggaggggtggccgccgccgaccaaagaagagattttatcgacatcgaaagtaaatttgccctaaggacccctgaagacacagctgaggacacttgccacctcattcccggagtagcagagtccgtggctacctgtcatttcaatcacagcagcaaaaccttcatggtgatccatggctggacggtaacaggaatgtatgagagttgggtgccaaaacttgtggccgccctgtacaagagagaaccagactccaatgtcattgtggtggactggctgtcacgggctcaggagcattacccagtgtccgcgggctacaccaaactggtgggacaggatgtggcccggtttatcaactggatggaggaggagtttaactaccctctggacaatgtccatctcttgggatacagccttggagcccatgctgctggcattgcaggaagtctgaccaataagaaagtcaacagaattactggcctcgatccagctggacctaactttgagtatgcagaagccccgagtcgtctttctcctgatgatgcagattttgtagacgtcttacacacattcaccagagggtcccctggtcgaagcattggaatccagaaaccagttgggcatgttgacatttacccgaatggaggtacttttcagccaggatgtaacattggagaagctatccgcgtgattgcagagagaggacttggagatgtggaccagctagtgaagtgctcccacgagcgctccattcatctcttcatcgactctctgttgaatgaagaaaatccaagtaaggcctacaggtgcagttccaaggaagcctttgagaaagggctctgcttgagttgtagaaagaaccgctgcaacaatctgggctatgagatcaataaagtcagagccaaaagaagcagcaaaatgtacctgaagactcgttctcagatgccctacaaagtcttccattaccaagtaaagattcatttttctgggactgagagtgaaacccataccaatcaggcctttgagatttctctgtatggcaccgtggccgagagtgagaacatcccattcactctgcctgaagtttccacaaataagacctactccttcctaatttacacagaggtagatattggagaactactcatgttgaagctcaaatggaagagtgattcatactttagctggtcagactggtggagcagtcccggcttcgccattcagaagatcagagtaaaagcaggagagactcagaaaaaggtgatcttctgttctagggagaaagtgtctcatttgcagaaaggaaaggcacctgcggtatttgtgaaatgccatgacaagtctctgaataagaagtcaggctgaaactgggcgaatctacagaacaaagaacggcatgtgaattctgtgaagaatgaagtggaggaagtaacttttacaaaacatacccagtgtttggggtgtttcaaaagtggattttcctgaatattaatcccagccctacccttgttagttattttaggagacagtctcaagcactaaaaagtggctaattcaatttatggggtatagtggccaaatagcacatcctccaacgttaaaagacagtggatcatgaaaagtgctgttttgtcctttgagaaagaaataattgtttgagcgcagagtaaaataaggctccttcatgtggcgtattgggccatagcctataattggttagaacctcctattttaattggaattctggatctttcggactgaggccttctcaaactttactctaagtctccaagaatacagaaaatgcttttccgcggcacgaatcagactcatctacacagcagtatgaatgatgttttagaatgattccctcttgctattggaatgtggtccagacgtcaaccaggaacatgtaacttggagagggacgaagaaagggtctgataaacacagaggttttaaacagtccctaccattggcctgcatcatgacaaagttacaaattcaaggagatataaaatctagatcaattaattcttaataggctttatcgtttattgcttaatccctctctcccccttcttttttgtctcaagattatattataataatgttctctgggtaggtgttgaaaatgagcctgtaatcctcagctgacacataatttgaatggtgcagaaaaaaaaaagataccgtaattttattattagattctccaaatgattttcatcaatttaaaatcattcaatatctgacagttactcttcagttttaggcttaccttggtcatgcttcagttgtacttccagtgcgtctcttttgttcctggctttgacatgaaaagataggtttgagttcaaattttgcattgtgtgagcttctacagattttagacaaggaccgtttttactaagtaaaagggtggagaggttcctggggtggattcctaagcagtgcttgtaaaccatcgcgtgcaatgagccagatggagtaccatgagggttgttatttgttgtttttaacaactaatcaagagtgagtgaacaactatttataaactagatctcctatttttcagaatgctcttctacgtataaatatgaaatgataaagatgtcaaatatctcagaggctatagctgggaacccgactgtgaaagtatgtgatatctgaacacatactagaaagctctgcatgtgtgttgtccttcagcataattcggaagggaaaacagtcgatcaagggatgtattggaacatgtcggagtagaaattgttcctgatgtgccagaacttcgaccctttctctgagagagatgatcgtgcctataaatagtaggaccaatgttgtgattaacatcatcaggcttggaatgaattctctctaaaaataaaatgatgtatgatttgttgttggcatcccctttattaattcattaaatttctggatttgggttgtgacccagggtgcattaacttaaaagattcactaaagcagcacatagcactgggaactctggctccgaaaaactttgttatatatatcaaggatgttctggctttacattttatttattagctgtaaatacatgtgtggatgtgtaaatggagcttgtacatattggaaaggtcattgtggctatctgcatttataaatgtgtggtgctaactgtatgtgtctttatcagtgatggtctcacagagccaactcactcttatgaaatgggctttaacaaaacaagaaagaaacgtacttaactgtgtgaagaaatggaatcagcttttaataaaattgacaacattttattaccac

SEQ ID NO: 14 - protein, 475 aa, Homo sapien
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 15 - DNA, 1466 bp, Homo sapien
  CDS (115)...(1305): Nucleotide sequence encoding apolipoprotein A-IV (APOA4)
agttcccactgcagcgcaggtgagctctcctgaggacctctctgtcagctcccctgattgtagggaggcatccagtgtggcaagaaactcctccagcccagcaagcagctcaggatgttcctgaaggccgtggtcctgaccctggccctggtggctgtcgccggagccagggctgaggtcagtgctgaccaggtggccacagtgatgtgggactacttcagccagctgagcaacaatgccaaggaggccgtggaacatctccagaaatctgaactcacccagcaactcaatgccctcttccaggacaaacttggagaagtgaacacttacgcaggtgacctgcagaagaagctggtgccctttgccaccgagctgcatgaacgcctggccaaggactcggagaaactgaaggaggagattgggaaggagctggaggagctgagggcccggctgctgccccatgccaatgaggtgagccagaagatcggggacaacctgcgagagcttcagcagcgcctggagccctacgcggaccagctgcgcacccaggtcaacacgcaggccgagcagctgcggcgccagctgaccccctacgcacagcgcatggagagagtgctgcgggagaacgccgacagcctgcaggcctcgctgaggccccacgccgacgagctcaaggccaagatcgaccagaacgtggaggagctcaagggacgccttacgccctacgctgacgaattcaaagtcaagattgaccagaccgtggaggagctgcgccgcagcctggctccctatgctcaggacacgcaggagaagctcaaccaccagcttgagggcctgaccttccagatgaagaagaacgccgaggagctcaaggccaggatctcggccagtgccgaggagctgcggcagaggctggcgcccttggccgaggacgtgcgtggcaacctgaggggcaacaccgaggggctgcagaagtcactggcagagctgggtgggcacctggaccagcaggtggaggagttccgacgccgggtggagccctacggggaaaacttcaacaaagccctggtgcagcagatggaacagctcaggacgaaactgggcccccatgcgggggacgtggaaggccacttgagcttcctggagaaggacctgagggacaaggtcaactccttcttcagcaccttcaaggagaaagagagccaggacaagactctctccctccctgagctggagcaacagcaggaacagcatcaggagcagcagcaggagcaggtgcagatgctggcccctttggagagctgagctgcccctggtgcactggccccaccctcgtggacacctgccctgccctgccacctgtctgtctgtcccaaagaagttctggtatgaacttgaggacacatgtccagtgggaggtgagaccacctctcaatattcaataaagctgctgagaatctagcctc

SEQ ID NO: 16 - protein, 396 aa, Homo sapien
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVNTQAEQLRRQLTPYAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLRGNTEGLQKSLAELGGHLDQQVEEFRRRVEPYGENFNKALVQQMEQLRTKLGPHAGDVEGHLSFLEKDLRDKVNSFFSTFKEKESQDKTLSLPELEQQQEQHQEQQQEQVQMLAPLES

SEQ ID NO: 17 - DNA, 1156 bp, Homo sapien
  CDS (61)...(1014): Nucleotide Sequence encoding apolipoprotein E (APOE)
cgcagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 18 - protein, 317 aa, Homo sapien [sequence omitted]

SEQ ID NO: 19 - DNA, 1603 bp, Homo sapien
  CDS (58)...(1557): Nucleotide sequence encoding hepatic lipase (LIPC)
ggtctctttggcttcagaaattaccaagaaagcctggaccccgggtgaaacggagaaatggacacaagtcccctgtgtttctccattctgttggttttatgcatctttatccaatcaagtgcccttggacaaagcctgaaaccagagccatttggaagaagagctcaagctgttgaaacaaacaaaacgctgcatgagatgaagaccagattcctgctctttggagaaaccaatcagggctgtcagattcgaatcaatcatccggacacgttacaggagtgcggcttcaactcctccctgcctctggtgatgataatccacgggtggtcggtggacggcgtgctagaaaactggatctggcagatggtggccgcgctgaagtctcagccggcccagccagtgaacgtggggctggtggactggatcaccctggcccacgaccactacaccatcgccgtccgcaacacccgccttgtgggcaaggaggtcgcggctcttctccggtggctggaggaatctgttcaactctctcgaagccatgttcacctaattgggtacagcctgggtgcacacgtgtcaggatttgccggcagttccatcggtggaacgcacaagattgggagaatcacagggctggatgccgcgggacctttgtttgagggaagtgcccccagcaatcgtctttctccagatgatgccaattttgtggatgccattcatacctttacgcgggagcacatgggcctgagcgtgggcatcaaacagcccataggacactatgacttctatcccaacgggggctccttccagcctggctgccacttcctagagctctacagacatattgcccagcacggcttcaatgccatcacccagaccataaaatgctcccacgagcgatcggtgcaccttttcatcgactccttgctgcacgccggcacgcagagcatggcctacccgtgtggtgacatgaacagcttcagccagggcctgtgcctgagctgcaagaagggccgctgcaacacgctgggctaccacgtccgccaggagccgcggagcaagagcaagaggctcttcctcgtaacgcgagcccagtcccccttcaaagtttatcattaccagttaaagatccagttcatcaaccaaactgagacgccaatacaaacaacttttaccatgtcactactcggaacaaaagagaaaatgcagaaaattcccatcactctgggcaaaggaattgctagtaataaaacgtattcctttcttatcacgctggatgtggatatcggcgagctgatcatgatcaagttcaagtgggaaaacagtgcagtgtgggccaatgtctgggacacggtccagaccatcatcccatggagcacagggccgcgccactcaggcctcgttctgaagacgatcagagtcaaagcaggagaaacccagcaaagaatgacattttgttcagaaaacacagatgacctactacttcgcccaacccaggaaaaaatcttcgtgaaatgtgaaataaagtctaaaacatcaaagcgaaagatcagatgagatttaatgaagacccagtgtaaagaataaatgaatcttactcctt

SEQ ID NO: 20 - protein, 499 aa, Homo sapien
MDTSPLCFSILLVLCIFIQSSALGQSLKPEPFGRRAQAVETNKTLHEMKTRFLLFGETNQGCQIRINHPDTLQECGFNSSLPLVMIIHGWSVDGVLENWIWQMVAALKSQPAQPVNVGLVDWITLAHDHYTIAVRNTRLVGKEVAALLRWLEESVQLSRSHVHLIGYSLGAHVSGFAGSSIGGTHKIGRITGLDAAGPLFEGSAPSNRLSPDDANFVDAIHTFTREHMGLSVGIKQPIGHYDFYPNGGSFQPGCHFLELYRHIAQHGFNAITQTIKCSHERSVHLFIDSLLHAGTQSMAYPCGDMNSFSQGLCLSCKKGRCNTLGYHVRQEPRSKSKRLFLVTRAQSPFKVYHYQLKIQFINQTETPIQTTFTMSLLGTKEKMQKIPITLGKGIASNKTYSFLITLDVDIGELIMIKFKWENSAVWANVWDTVQTIIPWSTGPRHSGLVLKTIRVKAGETQQRMTFCSENTDDLLLRPTQEKIFVKCEIKSKTSKRKIR

SEQ ID NO: 21 - DNA, 1346 bp, Homo sapien
  CDS (10)...(1077): Nucleotide sequence encoding paraoxonase 1 (PON1)
cccccgaccatggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacatggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaacagaccgatttgcacccatgccatagaaactgaggccattatttcaaccgcttgccatattccgaggacccagtgttcttagctgaacaatgaatgctgaccctaaatgtggacatcatgaagcatcaaagcactgtttaactgggagtgatatgatgtgtagggcttttttttgagaatacactatcaaatcagtcttggaatacttgaaaacctcatttaccataaaaatccttctcactaaaatggataaatcagttaaaaaaaaa

SEQ ID NO: 22 - protein, 355 aa, Homo sapien
MAKLIALTLLGMGLALFRNHQSSYQTRLNALREVQPVELPNCNLVKGIETGSEDMEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLQSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 23 - DNA, 1570 bp, Homo sapien
  CDS (1)...(1097): Nucleotide sequence encoding paraoxonase 2 (PON2)
cggagcgaggcagcgcgcccggctcccgcgccatggggcggctggtggctgtgggcttgctggggatcgcgctggcgctcctgggcgagaggcttctggcactcagaaatcgacttaaagcctccagagaagtagaatctgtagaccttccacactgccacctgattaaaggaattgaagctggctctgaagatattgacatacttcccaatggtctggctttttttagtgtgggtctaaaattcccaggactccacagctttgcaccagataagcctggaggaatactaatgatggatctaaaagaagaaaaaccaagggcacgggaattaagaatcagtcgtgggtttgatttggcctcattcaatccacatggcatcagcactttcatagacaacgatgacacagtttatctctttgttgtaaaccacccagaattcaagaatacagtggaaatttttaaatttgaagaagcagaaaattctctgttgcatctgaaaacagtcaaacatgagcttcttccaagtgtgaatgacatcacagctgttggaccggcacatttctatgccacaaatgaccactacttctctgatcctttcttaaagtatttagaaacatacttgaacttacactgggcaaatgttgtttactacagtccaaatgaagttaaagtggtagcagaaggatttgattcagcaaatgggatcaatatttcacctgatgataagtatatctatgttgctgacatattggctcatgaaattcatgttttggaaaaacacactaatatgaatttaactcagttgaaggtacttgagctggatacactggtggataatttatctattgatccttcctcgggggacatctgggtaggctgtcatcctaatggccagaagctcttcgtgtatgacccgaacaatcctccctcgtcagaggttctccgcatccagaacattctatctgagaagcctacagtgactacagtttatgccaacaatgggtctgttctccaaggaagttctgtagcctcagtgtatgatgggaagctgctcataggcactttataccacagagccttgtattgtgaactctaaattgtacttttggcatgaaagtgcgataacttaacaattaattttctatgaattgctaattctgagggaatttaaccagcaacattgacccagaaatgtatggcatgtgtagttaattttattccagtaaggaacggcccttttagttcttagagcacttttaacaaaaaaggaaaatgaacaggttctttaaaatgccaagcaagggacagaaaagaaagctgctttcgaataaagtgaatacattttgcacaaagtaagcctcacctttgccttccaactgccagaacatggattccactgaaatagagtgaattatatttccttaaaatgtgagtgacctcacttctggcactgtgactactatggctgtttagaactactgataacgtattttgatgttttgtacttacatctttgtttaccattaaaaagttggagttatattaaagactaactaaaatcccagtttt

SEQ ID NO: 24 - protein, 342 aa, Homo sapien
RSEAARPAPAPWGGWWLWACWGSRWRSWARGFWHSEIDLKPPEKNLTFHTATLKELKLALKILTYFPMVWLFLVWVNSQDSTALHQISLEEYWIKKKNQGHGNESVVGLIWPHSIHMASALSTTMTQFISLLTTQNSRIQWKFLNLKKQKILCCIKQSNMSFFQVMTSQLLDRHISMPQMTTTSLILSSIKHTTYTGQMLFTTVQMKLKWQKDLIQQMGSIFHLMISISMLLTYWLMKFMFWKNTLIILSRYLSWIHWWIIYLLILPRGTSGAVILMARSSSCMTRTILPRQRFSASRTFYLRSLQLQFMPTMGLFSKEVLPQCMMGSCSALYTTEPCIVNS

SEQ ID NO: 25 - DNA, 533 bp, Homo sapien
  CDS (47)...(346): Nucleotide sequence encoding apolipoprotein C-III(APOC3)
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 26 - protein, 99 aa, Homo sapien
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 27 - DNA, 8925 bp, Homo sapien
  misc_feature 5081, 5082, 5083, 5084, 5085, 5086, 5087, 5088, 5089,5090, 5091, 5092, 5093, 5094, 5095, 5096, 5097, 5098, 5099, 5100,5101, 5102, 5103, 5104, 5105, 5106, 5107, 5108, 5109, 5110,5111, 5112, 5113, 5114, 5115, 5116, 5117, 5118, 5119: n = A,T,C or G
  misc_feature 5120, 5121, 5122, 5123, 5124, 5125, 5126, 5127, 5128,5129, 5130, 5131, 5132, 5133, 5134, 5135, 5136, 5137, 5138, 5139,5140, 5141, 5142, 5143, 5144, 5145, 5146, 5147, 5148, 5149,5150, 5151, 5152, 5153, 5154, 5155, 5156, 5157, 5158: n = A,T,C or G
  misc_feature 5159, 5160, 5161, 5162, 5163, 5164, 5165, 5166, 5167,5168, 5169, 5170, 5171, 5172, 5173, 5174, 5175, 5176, 5177, 5178,5179: n = A,T,C or G
  CDS (5020)...(6162): Nucleotide encoding ATP-binding cassette (ABC1)
ctcagtgtcagctgctgctggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccaaaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctcggatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattaagttgtttccaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaatctttccttttcagtgctttgggctcctgggagttaatggggctggaaaatcatcaactttcaagatgttaacaggagataccactgttaccagaggagatgctttccttaacatttgcagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacacttttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctccaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccagaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtata

SEQ ID NO: 28 - protein, 380 aa, Homo sapien
  VARIANT 21, 22, 23, 24, 25, 26, 27, 28, 29, 30, 31, 32, 33,34, 35, 36, 37, 38, 39, 40, 41, 42, 43, 44, 45, 46, 47, 48, 49,50, 51, 52, 53, 54: Xaa = Any Amino Acid
SLSSTGSLILSGICAIKLFPXXXXXXXXXXXXXXXXXXXXXXXXXXXXXXXXXXIFPFQCFGLLGVNGAGKSSTFKMLTGDTTVTRGDAFLNICSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 29 - DNA, 897 bp, Homo sapien
  CDS (39)...(842): Nucleotide sequence encoding apolipoprotein A-1 (APOA1)
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 30 - protein, 267 aa, Homo sapien
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 31 - DNA, 14121 bp, Homo sapien
  CDS (129)...(13820): Nucleotide sequence encoding apolipoprotein B (APOB)
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaacaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctcaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctgaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtagtggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgatgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttgacccaccaatacaagttgaaggagactattcagaagctaagcaatgtcctacaacaagttaagataaaagattactttgagaaattggttggatttattgatgatgctgtgaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagactctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtcgaaatgaaagtaaagatcatcagaaccattgaccagatgcagaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccacatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaaaaaccacaaaagccgaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattactcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacacttgcacaccgtgacttcagtgcagaatatgaagaagatggcaaatttgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaaaaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcaatgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 32 - protein, 4563 aa, Homo sapien
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 33 - DNA, 2196 bp, Homo sapien
  CDS (13)...(1983): Nucleotide sequence encoding 5,10-methylenetetrahydofolate reductase (MTHFR)
aattccggagccatggtgaacgaagccagaggaaacagcagcctcaacccctgcttggagggcagtgccagcagtggcagtgagagctccaaagatagttcgagatgttccaccccgggcctggaccctgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagtgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagcaagtgtctttgaagtctttgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatgaccctgcgtcctgacgccctgcgttggagccactcctgtcccgccttcctcctccacagtgctgcttctcttgggaactccactctccttcgtgtctctcccaccccggcctccactcccccacctgacaatggcagctagactggagtgaggcttccaggctcttcctggacctgagtcggccccacatgggaacctagtactctctgctcta

SEQ ID NO: 34 - protein, 656 aa, Homo sapien
MVNEARGNSSLNPCLEGSASSGSESSKDSSRCSTPGLDPERHERLREKMRRRLESGDKWFSLEFFPPRTAEGAVNLISRFDRMAAGGPLYIDVTWHPAGDPGSDKETSSMMIASTAVNYCGLETILHMTCCRQRLEEITGHLHKAKQLGLKNIMALRGDPIGDQWEEEEGGFNYAVDLVKHIRSEFGDYFDICVAGYPKGHPEAGSFEADLKHLKEKVSAGADFIITQLFFEADTFFRFVKACTDMGITCPIVPGIFPIQGYHSLRQLVKLSKLEVPQEIKDVIEPIKDNDAAIRNYGIELAVSLCQELLASGLVPGLHFYTLNREMATTEVLKRLGMWTEDPRRPLPWALSAHPKRREEDVRPIFWASRPKSYIYRTQEWDEFPNGRWGNSSSPAFGELKDYYLFYLKSKSPKEELLKMWGEELTSEASVFEVFVLYLSGEPNRNGHKVTCLPWNDEPLAAETSLLKEELLRVNRQGILTINSQPNINGKPSSDPIVGWGPSGGYVFQKAYLEFFTSRETAEALLQVLKKYELRVNYHLVNVKGENITNAPELQPNAVTWGIFPGREIIQPTVVDPVSFMFWKDEAFALWIERWGKLYEEESPSRTIIQYIHDNYFLVNLVDNDFPLDNCLWQVVEDTLELLNRPTQNARETEAP

SEQ ID NO: 35 - DNA, 3834 bp, Homo sapien
  CDS (117)...(1949): Nucleotide sequence encoding selectin E (SELE)
cctgagacagaggcagcagtgatacccacctgagagatcctgtgtttgaacaactgcttcccaaaacggaaagtatttcaagcctaaacctttgggtgaaaagaactcttgaagtcatgattgcttcacagtttctctcagctctcactttggtgcttctcattaaagagagtggagcctggtcttacaacacctccacggaagctatgacttatgatgaggccagtgcttattgtcagcaaaggtacacacacctggttgcaattcaaaacaaagaagagattgagtacctaaactccatattgagctattcaccaagttattactggattggaatcagaaaagtcaacaatgtgtgggtctgggtaggaacccagaaacctctgacagaagaagccaagaactgggctccaggtgaacccaacaataggcaaaaagatgaggactgcgtggagatctacatcaagagagaaaaagatgtgggcatgtggaatgatgagaggtgcagcaagaagaagcttgccctatgctacacagctgcctgtaccaatacatcctgcagtggccacggtgaatgtgtagagaccatcaataattacacttgcaagtgtgaccctggcttcagtggactcaagtgtgagcaaattgtgaactgtacagccctggaatcccctgagcatggaagcctggtttgcagtcacccactgggaaacttcagctacaattcttcctgctctatcagctgtgataggggttacctgccaagcagcatggagaccatgcagtgtatgtcctctggagaatggagtgctcctattccagcctgcaatgtggttgagtgtgatgctgtgacaaatccagccaatgggttcgtggaatgtttccaaaaccctggaagcttcccatggaacacaacctgtacatttgactgtgaagaaggatttgaactaatgggagcccagagccttcagtgtacctcatctgggaattgggacaacgagaagccaacgtgtaaagctgtgacatgcagggccgtccgccagcctcagaatggctctgtgaggtgcagccattcccctgctggagagttcaccttcaaatcatcctgcaacttcacctgtgaggaaggcttcatgttgcagggaccagcccaggttgaatgcaccactcaagggcagtggacacagcaaatcccagtttgtgaagctttccagtgcacagccttgtccaaccccgagcgaggctacatgaattgtcttcctagtgcttctggcagtttccgttatgggtccagctgtgagttctcctgtgagcagggttttgtgttgaagggatccaaaaggctccaatgtggccccacaggggagtgggacaacgagaagcccacatgtgaagctgtgagatgcgatgctgtccaccagcccccgaagggtttggtgaggtgtgctcattcccctattggagaattcacctacaagtcctcttgtgccttcagctgtgaggagggatttgaattatatggatcaactcaacttgagtgcacatctcagggacaatggacagaagaggttccttcctgccaagtggtaaaatgttcaagcctggcagttccgggaaagatcaacatgagctgcagtggggagcccgtgtttggcactgtgtgcaagttcgcctgtcctgaaggatggacgctcaatggctctgcagctcggacatgtggagccacaggacactggtctggcctgctacctacctgtgaagctcccactgagtccaacattcccttggtagctggactttctgctgctggactctccctcctgacattagcaccatttctcctctggcttcggaaatgcttacggaaagcaaagaaatttgttcctgccagcagctgccaaagccttgaatcagacggaagctaccaaaagccttcttacatcctttaagttcaaaagaatcagaaacaggtgcatctggggaactagagggatacactgaagttaacagagacagataactctcctcgggtctctggcccttcttgcctactatgccagatgcctttatggctgaaaccgcaacacccatcaccacttcaatagatcaaagtccagcaggcaaggacggccttcaactgaaaagactcagtgttccctttcctactctcaggatcaagaaagtgttggctaatgaagggaaaggatattttcttccaagcaaaggtgaagagaccaagactctgaaatctcagaattccttttctaactctcccttgctcgctgtaaaatcttggcacagaaacacaatattttgtggctttctttcttttgcccttcacagtgtttcgacagctgattacacagttgctgtcataagaatgaataataattatccagagtttagaggaaaaaaatgactaaaaatattataacttaaaaaaatgacagatgttgaatgcccacaggcaaatgcatggagggttgttaatggtgcaaatcctactgaatgctctgtgcgagggttactatgcacaatttaatcactttcatccctatgggattcagtgcttcttaaagagttcttaaggattgtgatatttttacttgcattgaatatattataatcttccatacttcttcattcaatacaagtgtggtagggacttaaaaaacttgtaaatgctgtcaactatgatatggtaaaagttacttattctagattaccccctcattgtttattaacaaattatgttacatctgttttaaatttatttcaaaaagggaaactattgtcccctagcaaggcatgatgttaaccagaataaagttctgagtgtttttactacagttgttttttgaaaacatggtagaattggagagtaaaaactgaatggaaggtttgtatattgtcagatattttttcagaaatatgtggtttccacgatgaaaaacttccatgaggccaaacgttttgaactaataaaagcataaatgcaaacacacaaaggtataattttatgaatgtctttgttggaaaagaatacagaaagatggatgtgctttgcattcctacaaagatgtttgtcagatgtgatatgtaaacataattcttgtatattatggaagattttaaattcacaatagaaactcaccatgtaaaagagtcatctggtagatttttaacgaatgaagatgtctaatagttattccctatttgttttcttctgtatgttagggtgctctggaagagaggaatgcctgtgtgagcaagcatttatgtttatttataagcagatttaacaattccaaaggaatctccagttttcagttgatcactggcaatgaaaaattctcagtcagtaattgccaaagctgctctagccttgaggagtgtgagaatcaaaactctcctacacttccattaacttagcatgtgttgaaaaaaaaagtttcagagaagttctggctgaacactggcaacgacaaagccaacagtcaaaacagagatgtgataaggatcagaacagcagaggttcttttaaaggggcagaaaaactctgggaaataagagagaacaactactgtgatcaggctatgtatggaatacagtgttattttctttgaaattgtttaagtgttgtaaatatttatgtaaactgcattagaaattagctgtgtgaaataccagtgtggtttgtgtttgagttttattgagaattttaaattataacttaaaatattttataatttttaaagtatatatttatttaagcttatgtcagacctatttgacataacactataaaggttgacaataaatgtgcttatgttt

SEQ ID NO: 36 - protein, 610 aa, Homo sapien
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELYGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 37 - DNA, 1922 bp, Homo sapien
  CDS (406)...(1428): Nucleotide sequence encoding nucleotide binding protein (G Protein), beta polypeptide 3 (GNB3)
ccacaataggggcagacctgtccatccttctctgtgggtcccctgtacctttctcccccaacaggatcagacccagaggcagctggttggggtttgtcgagaagaaggattatccagatcagtcctttctaatctcagctcctgcctgtaccctcccatactcaccaaaccctcttccccaccaccctgagctgaggagcacagtttgaggcccccccaaccccccgccggtcggggccaggccaggccaggccagctcctctggcagcagagcctgggcaggtgacgggcgggcgcgggcgtcgcagctgagggagtaaggaggctcccaggaaccggagctggaaacccggccgaggtccagccagagcccaagagccagagtgacccctcgacctgtcagccatgggggagatggagcaactgcgtcaggaagcggagcagctcaagaagcagattgcagatgccaggaaagcctgtgctgacgttactctggcagagctggtgtctggcctagaggtggtgggacgagtccagatgcggacgcggcggacgttaaggggacacctggccaagatttacgccatgcactgggccactgattctaagctgctggtaagtgcctcgcaagatgggaagctgatcgtgtgggacagctacaccaccaacaaggtgcacgccatcccactgcgctcctcctgggtcatgacctgtgcctatgccccatcagggaactttgtggcatgtggggggctggacaacatgtgttccatctacaacctcaaatcccgtgagggcaatgtcaaggtcagccgggagctttctgctcacacaggttatctctcctgctgccgcttcctggatgacaacaatattgtgaccagctcgggggacaccacgtgtgccttgtgggacattgagactgggcagcagaagactgtatttgtgggacacacgggtgactgcatgagcctggctgtgtctcctgacttcaatctcttcatttcgggggcctgtgatgccagtgccaagctctgggatgtgcgagaggggacctgccgtcagactttcactggccacgagtcggacatcaacgccatctgtttcttccccaatggagaggccatctgcacgggctcggatgacgcttcctgccgcttgtttgacctgcgggcagaccaggagctgatctgcttctcccacgagagcatcatctgcggcatcacgtccgtggccttctccctcagtggccgcctactattcgctggctacgacgacttcaactgcaatgtctgggactccatgaagtctgagcgtgtgggcatcctctctggccacgataacagggtgagctgcctgggagtcacagctgacgggatggctgtggccacaggttcctgggacagcttcctcaaaatctggaactgaggaggctggagaaagggaagtggaaggcagtgaacacactcagcagccccctgcccgaccccatctcattcaggtgttctcttctatattccgggtgccattcccactaagctttctcctttgagggcagtggggagcatgggactgtgcctttgggaggcagcatcagggacacaggggcaaagaactgccccatctcctcccatggccttccctccccacagtcctcacagcctctcccttaatgagcaaggacaacctgcccctccccagccctttgcaggcccagcagacttgagtctgaggccccaggccctaggattcctcccccagagccactacctttgtccaggcctgggtggtatagggcgtttggccctgtgactatggctctggcaccactagggtcctggccctcttcttattcatgctttctcctttttctacctttttttctctcctaagacacctgcaataaagtgtagcaccctggt

SEQ ID NO: 38 - protein, 340 aa, Homo sapien [sequence omitted]

SEQ ID NO: 39 - DNA, 2443 bp, Homo sapien
  CDS (162)...(1253): Nucleotide sequence encoding angiotensin receptor 2  (AGTR2)
acgtcccagcgtctgagagaacgagtaagcaagaattcaaagcattctgcagcctgaattttgaaggagtgtgtttaggcactaagcaagctgatttatgataactgctttaaacttcaacaaccaaaggcataagaactaggagctgctgacatttcaatatgaagggcaactccacccttgccactactagcaaaaacattaccagcggtcttcacttcgggcttgtgaacatctctggcaacaatgagtctaccttgaactgttcacagaaaccatcagataagcatttagatgcaattcctattctttactacattatatttgtaattggatttctggtcaatattgtcgtggttacactgttttgttgtcaaaagggtcctaaaaaggtttctagcatatacatcttcaacctcgctgtggctgatttactccttttggctactcttcctctatgggcaacctattattcttatagatatgactggctctttggacctgtgatgtgcaaagtttttggttcttttcttaccctgaacatgtttgcaagcattttttttatcacctgcatgagtgttgataggtaccaatctgtcatctacccctttctgtctcaaagaagaaatccctggcaagcatcttatatagttccccttgtttggtgtatggcctgtttgtcctcattgccaacattttattttcgagacgtcagaaccattgaatacttaggagtgaatgcttgcattatggctttcccacctgagaaatatgcccaatggtcagctgggattgccttaatgaaaaatatccttggttttattatccctttaatattcatagcaacatgctattttggaattagaaaacacttactgaagacgaatagctatgggaagaacaggataacccgtgaccaagtcctgaagatggcagctgctgttgttctggccttcatcatttggtgccttcccttccatgttctgaccttcctggatgctctggcctggatgggtgtcattaatagctgcgaagttatagcagtcattgacctggcacttccttttgccatcctcttgggattcaccaacagctgcgttaatccgtttctgtattgttttgttggaaaccggttccaacagaagctccgcagtgtgtttagggttccaattacttggctccaagggaaaagagagagtatgtcttgccggaaaagcagttctcttagagaaatggagacctttgtgtcttaaacggagagcaaaatgcatgtaatcaacatggctacttgctttgaggctcaccagaattatttttaagtggttttaataaaataataaaatttcccctaatcttttctgaatcttctgaaaccaaatgtaactatgtttatcgtccagtgactttcaggaatgcccattgttttctgatatgtttgtacaagatttcattggtgagacatatttacaacctagaagtaactggtgatatatctcaaattgtaattaataatagattgtgaataatgatttggggattcagatttctctttgaaacatgcttgtgtttcttagtggggttttatatccatttttatcaggatttcctcttgaaccagaaccagtctttcaactcattgcatcatttacaagacaacattgtaagagagatgagcacttctaagttgagtatattataatagattagtactggattattcaggctttaggcatatgcttctttaaaaacgctataaattatattcctcttgcatttcacttgagtggaggtttatagttaatctataactacatattgaatagggctaggaatatagattaaatcatactcctatgctttagcttatttttacagttatagaaagcaagatgtactataacatagaattgcaatctataatatttgtgtgttcactaaactctgaataagcactttttaaaaaactttctactcattttaatgattgtttaaaggtttctattttctctgatacttttttgaaatcagtaaacactgtgtattgttgtaaaatgtaaaggtcacttttcacatccttgactttttagatgtgctgctttgatatataggacattgatttgatttttattattaatgctttggttctgggttgtttcctaaaatatctgggtggcttaaaaaaaactctttaacttgtaataaacccttaactggcataggaaatggtatccagaatggaattttgctacatggggtctgggtgggggcaaagagacccagtcaattacatgtttggtaccaagaaaggaacctgtcagggcagtacaatgtgactttgaaaatatataccgtgggggtagttttaccctatatctataaacactgtttgttccagaatctgtatgattctatggagctattttaaaccaattgcaggtctaga

SEQ ID NO: 40 - protein, 363 aa, Homo sapien [sequence omitted]

SEQ ID NO: 41 - DNA, 20 bp, Artificial Sequence
  Primer
actgcctgataaccatgctg

SEQ ID NO: 42 - DNA, 20 bp, Artificial Sequence
  Primer
atacttacacaccaggaggg

SEQ ID NO: 43 - DNA, 19 bp, Artificial Sequence
  Primer
atgcctgctccaaaggcac

SEQ ID NO: 44 - DNA, 20 bp, Artificial Sequence
  Primer
atgcctgctccaaaggcacc

SEQ ID NO: 45 - DNA, 21 bp, Artificial Sequence
  Primer
atgcctgctccaaaggcacat

SEQ ID NO: 46 - DNA, 20 bp, Artificial Sequence
  Primer
tacttctggttctctgagcg

SEQ ID NO: 47 - DNA, 20 bp, Artificial Sequence
  Primer
actcaccttgaactcgtctc

SEQ ID NO: 48 - DNA, 20 bp, Artificial Sequence
  Primer
tggttctctgagcgagtctt

SEQ ID NO: 49 - DNA, 21 bp, Artificial Sequence
  Primer
tggttctctgagcgagtcttc

SEQ ID NO: 50 - DNA, 22 bp, Artificial Sequence
  Primer
tggttctctgagcgagtctttc

SEQ ID NO: 51 - DNA, 20 bp, Artificial Sequence
  Primer
tgcagatggactttggcttc

SEQ ID NO: 52 - DNA, 20 bp, Artificial Sequence
  Primer
tgcttgccttctgctacaag

SEQ ID NO: 53 - DNA, 19 bp, Artificial Sequence
  Primer
cttccctgagcacctgctg

SEQ ID NO: 54 - DNA, 21 bp, Artificial Sequence
  Primer
cttccctgagcacctgctggt

SEQ ID NO: 55 - DNA, 20 bp, Artificial Sequence
  Primer
cttccctgagcacctgctga

SEQ ID NO: 56 - DNA, 20 bp, Artificial Sequence
  Primer
aacagctcaggacgaaactg

SEQ ID NO: 57 - DNA, 20 bp, Artificial Sequence
  Primer
agaaggagttgaccttgtcc

SEQ ID NO: 58 - DNA, 19 bp, Artificial Sequence
  Primer
ggaagctcaagtggccttc

SEQ ID NO: 59 - DNA, 20 bp, Artificial Sequence
  Primer
ggaagctcaagtggccttcc

SEQ ID NO: 60 - DNA, 22 bp, Artificial Sequence
  Primer
ggaagctcaagtggccttcaac

SEQ ID NO: 61 - DNA, 19 bp, Artificial Sequence
  Primer
aagtcactggcagagctgg

SEQ ID NO: 62 - DNA, 20 bp, Artificial Sequence
  Primer
gcaccagggctttgttgaag

SEQ ID NO: 63 - DNA, 19 bp, Artificial Sequence
  Primer
ttttccccgtagggctcca

SEQ ID NO: 64 - DNA, 20 bp, Artificial Sequence
  Primer
ttttccccgtagggctccac

SEQ ID NO: 65 - DNA, 21 bp, Artificial Sequence
  Primer
ttttccccgtagggctccagc

SEQ ID NO: 66 - DNA, 20 bp, Artificial Sequence
  Primer
tgcagaagtcactggcagag

SEQ ID NO: 67 - DNA, 20 bp, Artificial Sequence
  Primer
gttgaagttttccccgtagg

SEQ ID NO: 68 - DNA, 19 bp, Artificial Sequence
  Primer
actcctccacctgctggtc

SEQ ID NO: 69 - DNA, 20 bp, Artificial Sequence
  Primer
actcctccacctgctggtcc

SEQ ID NO: 70 - DNA, 21 bp, Artificial Sequence
  Primer
actcctccacctgctggtcta

SEQ ID NO: 71 - DNA, 20 bp, Artificial Sequence
  Primer
aggacgtgcgtggcaacctg

SEQ ID NO: 72 - DNA, 20 bp, Artificial Sequence
  Primer
agctctgccagtgacttctg

SEQ ID NO: 73 - DNA, 19 bp, Artificial Sequence
  Primer
gtgacttctgcagcccctc

SEQ ID NO: 74 - DNA, 20 bp, Artitifical sequence
gtgacttctgcagcccctca

SEQ ID NO: 75 - DNA, 22 bp, Artificial Sequence
  Primer
gtgacttctgcagcccctcggt

SEQ ID NO: 76 - DNA, 19 bp, Artificial Sequence
  Primer
cctgaccttccagatgaag

SEQ ID NO: 77 - DNA, 19 bp, Artificial Sequence
  Primer
tcaggttgccacgcacgtc

SEQ ID NO: 78 - DNA, 18 bp, Artificial Sequence
  Primer
caggatctcggccagtgc

SEQ ID NO: 79 - DNA, 19 bp, Artificial Sequence
  Primer
caggatctcggccagtgcc

SEQ ID NO: 80 - DNA, 20 bp, Artificial Sequence
  Primer
caggatctcggccagtgctg

SEQ ID NO: 81 - DNA, 20 bp, Artificial Sequence
  Primer
acctgcgagagcttcagcag

SEQ ID NO: 82 - DNA, 20 bp, Artificial Sequence
  Primer
tctccatgcgctgtgcgtag

SEQ ID NO: 83 - DNA, 18 bp, Artificial Sequence
  Primer
agctgcgcacccaggtca

SEQ ID NO: 84 - DNA, 19 bp, Artificial Sequence
  Primer
agctgcgcacccaggtcaa

SEQ ID NO: 85 - DNA, 20 bp, Artificial Sequence
  Primer
agctgcgcacccaggtcagc

SEQ ID NO: 86 - DNA, 19 bp, Artificial Sequence
  Primer
tgtccaaggagctgcaggc

SEQ ID NO: 87 - DNA, 20 bp, Artificial Sequence
  Primer
cttacgcagcttgcgcaggt

SEQ ID NO: 88 - DNA, 18 bp, Artificial Sequence
  Primer
gcggacatggaggacgtg

SEQ ID NO: 89 - DNA, 19 bp, Artificial Sequence
  Primer
gcggacatggaggacgtgc

SEQ ID NO: 90 - DNA, 20 bp, Artificial Sequence
  Primer
gcggacatggaggacgtgtg

SEQ ID NO: 91 - DNA, 20 bp, Artificial Sequence
  Primer
gttgtagaaagaaccgctgc

SEQ ID NO: 92 - DNA, 20 bp, Artificial Sequence
  Primer
gagaacgagtcttcaggtac

SEQ ID NO: 93 - DNA, 21 bp, Artificial Sequence
  Primer
acaatctgggctatgagatca

SEQ ID NO: 94 - DNA, 22 bp, Artificial Sequence
  Primer
acaatctgggctatgagatcaa

SEQ ID NO: 95 - DNA, 23 bp, Artificial Sequence
  Primer
acaatctgggctatgagatcagt

SEQ ID NO: 96 - DNA, 20 bp, Artificial Sequence
  Primer
cactctacactgcatgtctc

SEQ ID NO: 97 - DNA, 20 bp, Artificial Sequence
  Primer
acccttctgaaaaggagagg

SEQ ID NO: 98 - DNA, 20 bp, Artificial Sequence
  Primer
gaggagagacaaggcagata

SEQ ID NO: 99 - DNA, 21 bp, Artificial Sequence
  Primer
gaggagagacaaggcagatat

SEQ ID NO: 100 - DNA, 22 bp, Artificial Sequence
  Primer
gaggagagacaaggcagatagt

SEQ ID NO: 101 - DNA, 20 bp, Artificial Sequence
  Primer
aaaggttcagttgctgctgc

SEQ ID NO: 102 - DNA, 20 bp, Artificial Sequence
  Primer
gctggggaaggtctaataac

SEQ ID NO: 103 - DNA, 19 bp, Artificial Sequence
  Primer
gttgctgctgcctcgaatc

SEQ ID NO: 104 - DNA, 20 bp, Artificial Sequence
  Primer
gttgctgctgcctcgaatcc

SEQ ID NO: 105 - DNA, 21 bp, Artificial Sequence
  Primer
gttgctgctgcctcgaatctg

SEQ ID NO: 106 - DNA, 20 bp, Artificial Sequence
  Primer
cgtctttctccagatgatgc

SEQ ID NO: 107 - DNA, 20 bp, Artificial Sequence
  Primer
agtgtcctatgggctgtttg

SEQ ID NO: 108 - DNA, 21 bp, Artificial Sequence
  Primer
ggatgccattcatacctttac

SEQ ID NO: 109 - DNA, 22 bp, Artificial Sequence
  Primer
ggatgccattcatacctttacc

SEQ ID NO: 110 - DNA, 23 bp, Artificial Sequence
  Primer
ggatgccattcatacctttacgc

SEQ ID NO: 111 - DNA, 20 bp, Artificial Sequence
  Primer
tgggaaaacagtgcagtgtg

SEQ ID NO: 112 - DNA, 20 bp, Artificial Sequence
  Primer
tgatcgtcttcagaacgagg

SEQ ID NO: 113 - DNA, 23 bp, Artificial Sequence
  Primer
ccagaccatcatcatcccatgga

SEQ ID NO: 114 - DNA, 21 bp, Artificial Sequence
  Primer
ccagaccatcatcccatggaa

SEQ ID NO: 115 - DNA, 22 bp, Artificial Sequence
  Primer
ccagaccatcatcccatggagc

SEQ ID NO: 116 - DNA, 20 bp, Artificial Sequence
  Primer
cagcaatcgtctttctccag

SEQ ID NO: 117 - DNA, 20 bp, Artificial Sequence
  Primer
tcctatgggctgtttgatgc

SEQ ID NO: 118 - DNA, 21 bp, Artificial Sequence
  Primer
gtctttctccagatgatgcca

SEQ ID NO: 119 - DNA, 22 bp, Artificial Sequence
  Primer
gtctttctccagatgatgccaa

SEQ ID NO: 120 - DNA, 23 bp, Artificial Sequence
  Primer
gtctttctccagatgatgccagt

SEQ ID NO: 121 - DNA, 23 bp, Artificial Sequence
  Primer
agcggataacaatttcacacagg

SEQ ID NO: 122 - DNA, 16 bp, Artificial Sequence
  Primer
ggcgcacgcctccacg

(19) United States
(12) Patent Application Publication
(10) Pub. No.: US 2003/0224418 A1
(43) Pub. Date: Dec. 4, 2003

(54) Genes and polymorphisms associated with cardiovascular disease and their use

(76) Inventors: Andreas Braun, San Diego, CA (US); Patrick W. Kleyn, Concord, MA (US); Aruna Bansal, Landbeach (GB)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/403,902

(22) Filed: Mar. 27, 2003

Related U.S. Application Data
(62) Division of application No. 09/802,640, filed on Mar. 9, 2001, now abandoned.

(51) Int. Cl. C12Q 1/68

(52) U.S. Cl. 435/6

(57) ABSTRACT

Genes and polymorphisms associated with cardiovascular disease, methods that use the polymorphism to detect a predisposition to developing high cholesterol, low HDL or cardiovascular disease, to profile the response of subjects to therapeutic drugs and to develop therapeutic drugs are provided.

### RELATED APPLICATIONS

This application is a divisional application of copending U.S. patent application Ser. No. 09/802,640, filed Mar. 9, 2001, to Andreas Braun, Aruna Bansal and Patrick Kleyn, entitled “GENES AND POLYMORPHISMS ASSOCIATED WITH CARDIOVASCULAR DISEASE AND THEIR USE.” The benefit of priority to this application is claimed and the subject matter of the application is incorporated herein in its entirety.

### FIELD OF THE INVENTION

The field of the invention involves genes and polymorphisms of these genes that are associated with development of cardiovascular disease. Methods that use polymorphic markers for prognosticating, profiling drug response and drug discovery are provided.

### BACKGROUND OF THE INVENTION

Diseases in all organisms have a genetic component, whether inherited or resulting from the body's response to environmental stresses, such as viruses and toxins. The ultimate goal of ongoing genomic research is to use this information to develop new ways to identify, treat and potentially cure these diseases. The first step has been to screen disease tissue and identify genomic changes at the level of individual samples. The identification of these “disease” markers has then fueled the development and commercialization of diagnostic tests that detect these errant genes or polymorphisms. With the increasing numbers of genetic markers, including single nucleotide polymorphisms (SNPs), microsatellites, tandem repeats, newly mapped introns and exons, the challenge to the medical and pharmaceutical communities is to identify genotypes which not only identify the disease but also follow the progression of the disease and are predictive of an organism's response to treatment.

Polymorphisms

Polymorphisms have been known since 1901 with the identification of blood types. In the 1950's they were identified on the level of proteins using large population genetic studies. In the 1980's and 1990's many of the known protein polymorphisms were correlated with genetic loci on genomic DNA. For example, the gene dose of the apolipoprotein E type 4 allele was correlated with the risk of Alzheimer's disease in late onset families (see, e.g., Corder et al. (1993) Science 261: 921-923; mutation in blood coagulation factor V was associated with resistance to activated protein C (see, e.g., Bertina et al. (1994) Nature 369:64-67); resistance to HIV-1 infection has been shown in Caucasian individuals bearing mutant alleles of the CCR-5 chemokine receptor gene (see, e.g., Samson et al. (1996) Nature 382:722-725); and a hypermutable tract in antigen presenting cells (APC, such as macrophages), has been identified in familial colorectal cancer in individuals of Ashkenzi jewish background (see, e.g., Laken et al. (1997) Nature Genet. 17:79-83). There may be more than three million polymorphic sites in the human genome. Many have been identified, but not yet characterized or mapped or associated with a disease. Polymorphisms of the genome can lead to altered gene function, protein function or mRNA instability. To identify hose polymorphisms that have clinical relevance is the goal of a world-wide scientific effort. Discovery of such polymorphisms will have a fundamental impact on the identification and development of diagnostics and drug discovery.

Single Nucleotide Polymorphisms (SNPs)

Much of the focus of genomics has been in the identification of SNPs, which are important for a variety of reasons. They allow indirect testing (association of haplotypes) and direct testing (functional variants). They are the most abundant and stable genetic markers. Common diseases are best explained by common genetic alterations, and the natural variation in the human population aids in understanding disease, therapy and environmental interactions.

The organization of SNPs in the primary sequence of a gene into one of the limited number of combinations that exist as units of inheritance is termed a haplotype. Each haplotype therefore contains significantly more information than individual unorganized polymorphisms and provides an accurate measurement of the genomic variation in the two chromosomes of an individual. While it is well-established that many diseases are associated with specific variation in gene sequences and there are examples in which individual polymorphisms act as genetic markers for a particular phenotype, in other cases an individual polymorphism may be found in a variety of genomic backgrounds and therefore shows no definitive coupling between the polymorphism and the phenotype. In these instances, the observed haplotype and its frequency of occurrence in various genotypes will provide a better genetic marker for the phenotype.

Although risk factors for the development of cardiovascular disease are known, such as high serum cholesterol levels and low serum high density lipoprotein (HDL) levels, the genetic basis for the manifestation of these phenotypes remains unknown. An understanding of the genes that are responsible for controlling cholesterol and HDL levels, along with useful genetic markers and mutations in these genes that affect these phenotypes, will allow for detection of a predisposition for these risk factors and/or cardiovascular disease and the development of therapeutics to modulate such alterations. Therefore, it is an object herein to provide methods for using polymorphic markers to detect a predisposition to the manifestation of high serum cholesterol, low serum HDL and cardiovascular disease. The ultimate goals are the elucidation of pathological pathways, developing new diagnostic assays, determining genetic profiles for positive responses to therapeutic drugs, identifying new potential drug targets and identifying new drug candidates.

### SUMMARY OF THE INVENTION

A database of twins was screened for individuals which exhibit high or low levels of serum cholesterol or HDL. Using a full genome scanning approach, SNPs present in DNA samples from these individuals were examined for alleles that associate with either high levels of cholesterol or low levels of HDL. This lead to the discovery of the association of the cytochrome C oxidase subunit VIb (COX6B) gene and the N-acetylglucosaminyl transferase component GPI-1 (GPI-1) gene with these risks factors for developing cardiovascular disease. Specifically, a previously undetermined association of an allelic variant at nucleotide 86 of the COX6B gene and high serum cholesterol levels has been discovered. In addition, it has been discovered that an allelic variant at nucleotide 2577 of the GPI-1 gene is associated with low serum HDL levels. There was no previously known association between these two genes and risk factors related to cardiovascular disease.

Methods are provided for detecting the presence or absence of at least one allelic variant associated with high cholesterol, low HDL and/or cardiovascular disease by detecting the presence or absence of at least one allelic variant of the COX6B gene or the GPI-1 gene, individually or in combination with one or more allelic variants of other genes associated with cardiovascular disease.

Also provided are methods for indicating a predisposition to manifesting high serum cholesterol, low serum HDL and/or cardiovascular disease based on detecting the presence or absence of at least one allelic variant of the COX6B or GPI-1 genes, alone or in combination with one or more allelic variants of other genes associated with cardiovascular disease. These methods, referred to as haplotyping, are based on assaying more than one polymorphism of the COX6B and/or GPI-1 genes. One or more polymorphisms of other genes associated with cardiovascular disease may also be assayed at the same time. A collection of allelic variants of one or more genes may be more informative than a single allelic variant of any one gene. A single polymorphism of a collection of polymorphisms present in the COX6B and/or GPI-1 genes and in other genes associated with cardiovascular disease may be assayed individually or the collection may be assayed simultaneously using a multiplex assay method.

Also provided are microarrays comprising a probe selected from among an oligonucleotide complementary to a polymorphic region surrounding position 86 of the sense strand of the COX6B gene coding sequence; an oligonucleotide complementary to a polymorphic region surrounding the position of the antisense strand of COX6B corresponding to position 86 of the sense strand of the COX6B gene coding sequence; an oligonucleotide complementary to a polymorphic region surrounding position 2577 of the sense strand of the GPI-1 gene; and an oligonucleotide complementary to a polymorphic region surrounding the position of the antisense strand of GPI-1 corresponding to position 2577 of the sense strand of the GPI-1 gene. Microarrays are well known and can be made, for example, using methods set forth in U.S. Pat. Nos. 5,837,832; 5,858,659; 6,043,136; 6,043,031 and 6,156,501.

Further provided are methods of utilizing allelic variants of the COX6B or GPI-1 gene individually or together with one or more allelic variants of other genes associated with cardiovascular disease to predict a subject's response to a biologically active agent that modulates serum cholesterol, serum HDL, or a cardiovascular drug.

Also provided are methods to screen candidate biologically active agents for modulation of cholesterol, HDL or other factors associated with cardiovascular disease. These methods utilize cells or transgenic animals containing one or more allelic variants of the COX6B gene and/or the GPI-1 gene alone or in combination with allelic variants of one or more other genes associated with cardiovascular disease. Such animals should exhibit high cholesterol, low HDL or other known phenotypes associated with cardiovascular disease. Also, provided are methods to construct transgenic animals that are useful as models for cardiovascular disease by using one or more allelic variants of the COX6B gene and/or the GPI-1 gene alone or in combination with allelic variants of one or more other genes associated with cardiovascular disease.

Further provided are combinations of probes and primers and kits for predicting a predisposition to high serum cholesterol, low HDL levels and/or cardiovascular disease. In particular, combinations and kits comprise probes or primers which are capable of hybridizing adjacent to or at polymorphic regions of the COX6B and/or GPI-1 gene. The combinations and kits can also contain probes or primers which are capable of hybridizing adjacent to or at polymorphic regions of other genes associated with cardiovascular disease. The kits also optionally contain instructions for carrying out assays, interpreting results and for aiding in diagnosing a subject as having a predisposition towards developing high serum cholesterol, low HDL levels and/or cardiovascular disease. Combinations and kits are also provided for predicting a subject's response to a therapeutic agent directed toward modulating cholesterol, HDL, or another phenotype associated with cardiovascular disease. Such combinations and kits comprise probes or primers as described above.

In particular for the methods, combinations, kits and arrays described above, the polymorphisms are SNPs. The detection or identification is of a T nucleotide at position 86 of the sense strand of the COX6B gene coding sequence or the detection or identification of an A nucleotide at the corresponding position in the antisense strand of the COX6B gene coding sequence. Also embodied is the detection or identification of an A nucleotide at position 2577 of the sense strand of the GPI-1 gene or the detection or identification of a T nucleotide at the corresponding position in the antisense strand of the GPI-1 gene. In addition to the SNPs discussed above, other polymorphisms of the COX6B and GPI-1 genes can be assayed for association with high cholesterol or low HDL, respectively, and utilized as disclosed above.

Other genes containing allelic variants associated with high serum cholesterol, low HDL and/or cardiovascular disease, include, but are not limited to: cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase, E-selectin, G protein beta 3 subunit, and angiotensin II type 1 receptor gene.

The detection of the presence or absence of an allelic variant can utilize, but are not limited to, methods such as allele specific hybridization, primer specific extension, oligonucleotide ligation assay, restriction enzyme site analysis and single-stranded conformation polymorphism analysis.

In particular, primers utilized in primer specific extension hybridize adjacent to nucleotide 86 of the COX6B gene or nucleotide 2577 of the GPI-1 gene or the corresponding positions on the antisense strand (numbers refer to GenBank sequences, see pages 15-17). A primer can be extended in the presence of at least one dideoxynucleotide, particularly ddG, or two dideoxynucleotides, particularly ddG and ddC. Preferably, detection of extension products is by mass spectrometry. Detection of allelic variants can also involve signal moieties such as radioisotopes, enzymes, antigens, antibodies, spectrophotometric reagents, chemiluminescent reagents, fluorescent reagents and other light producing reagents.

Other probes and primers useful for the detection of allelic variants include those which hybridize at or adjacent to the SNPs described in Tables 1-3 and specifically those that comprise SEQ ID NOs.: 5, 10, 43, 48, 53, 58, 63, 68, 73, 78, 83, 88, 93, 98, 103, 108, 113, and 118.

### DESCRIPTION OF THE DRAWINGS

FIG. 1 depicts the allelic frequency and genotype for pools and individually determined samples of blood from individuals having low cholesterol levels and those with high cholesterol levels.

FIG. 2 depicts the allelic frequency and genotype for pools and individually determined samples of blood from individuals having high HDL levels and those with low HDL levels.

### DETAILED DESCRIPTION OF PREFERRED EMBODIMENTS

A. Definitions

Unless defined otherwise, all technical and scientific terms used herein have the same meaning as is commonly understood by one of skill in the art to which this invention belongs. All patents, patent applications and publications referred to throughout the disclosure herein are, unless noted otherwise, incorporated by reference in their entirety. In the event that there are a plurality of definitions for terms herein, those in this section prevail.

As used herein, sequencing refers to the process of determining a nucleotide sequence and can be performed using any method known to those of skill in the art. For example, if a polymorphism is identified or known, and it is desired to assess its frequency or presence in nucleic acid samples taken from the subjects that comprise the database, the region of interest from the samples can be isolated, such as by PCR or restriction fragments, hybridization or other suitable method known to those of skill in the art, and sequenced. For purposes herein, sequencing analysis is preferably effected using mass spectrometry (see, e.g., U.S. Pat. Nos. 5,547,835, 5,622,824, 5,851,765, and 5,928,906). Nucleic acids can also be sequenced by hybridization (see, e.g., U.S. Pat. Nos. 5,503,980, 5,631,134, 5,795,714) and including analysis by mass spectrometry (see, U.S. application Ser. Nos. 08/419,994 and 09/395,409). Alternatively, sequencing may be performed using other known methods, such as set forth in U.S. Pat. Nos. 5,525,464; 5,695,940; 5,834,189; 5,869,242; 5,876,934; 5,908,755; 5,912,118; 5,952,174; 5,976,802; 5,981,186; 5,998,143; 6,004,744; 6,017,702; 6,018,041; 6,025,136; 6,046,005; 6,087,095; 6,117,634, 6,013,431, WO 98/30883; WO 98/56954; WO 99/09218; WO/00/58519, and the others.

As used herein, “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides in length.

As used herein, “polymorphic gene” refers to a gene having at least one polymorphic region.

As used herein, “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

As used herein, the term “subject” refers to mammals and in particular human beings.

As used herein, the term “gene” or “recombinant gene” refers to a nucleic acid molecule comprising an open reading frame and including at least one exon and (optionally) at least one intron sequence. A gene can be either RNA or DNA. Genes may include regions preceding and following the coding region (leader and trailer).

As used herein, “intron” refers to a DNA sequence present in a given gene which is spliced out during mRNA maturation.

As used herein, the term “coding sequence” refers to that portion of a gene that encodes an amino acid sequence of a protein.

As used herein, the term “sense strand” refers to that strand of a double-stranded nucleic acid molecule that encodes the sequence of the mRNA that encodes the amino acid sequence encoded by the double-stranded nucleic acid molecule.

As used herein, the term “antisense strand” refers to that strand of a double-stranded nucleic acid molecule that is the complement of the sequence of the mRNA that encodes the amino acid sequence encoded by the double-stranded nucleic acid molecule.

As used herein, a DNA or nucleic acid homolog refers to a nucleic acid that includes a preselected conserved nucleotide sequence. By the term “substantially homologous” is meant having at least 80%, preferably at least 90%, most preferably at least 95% homology therewith or a less percentage of homology or identity and conserved biological activity or function.

Regarding hybridization, as used herein, stringency conditions to achieve specific hybridization refer to the washing conditions for removing the non-specific probes or primers and conditions that are equivalent to either high, medium, or low stringency as described below:

| | |
|---|---|
| 1) high stringency: | 0.1 × SSPE, 0.1% SDS, 65° C. |
| 2) medium stringency: | 0.2 × SSPE, 0.1% SDS, 50° C. |
| 3) low stringency: | 1.0 × SSPE, 0.1% SDS, 50° C. |

It is understood that equivalent stringencies may be achieved using alternative buffers, salts and temperatures.

As used herein, “heterologous DNA” is DNA that encodes RNA and proteins that are not normally produced in vivo by the cell in which it is expressed or that mediates or encodes mediators that alter expression of endogenous DNA by affecting transcription, translation, or other regulatable biochemical processes or is not present in the exact orientation or position as the counterpart DNA in a wildtype cell. Heterologous DNA may also be referred to as foreign DNA. Any DNA that one of skill in the art would recognize or consider as heterologous or foreign to the cell in which is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

As used herein, a “promoter region” refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. For gene expression a DNA sequence and a regulatory sequence(s) are connected in such a way to control or permit gene expression when the appropriate molecular, e.g., transcriptional activator proteins, are bound to the regulatory sequence(s).

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. “Plasmid” and “vector” are used interchangeably as the plasmid is the most commonly used form of vector. Also included are other forms of expression vectors that serve equivalent functions and that become known in the art subsequently hereto.

As used herein, “indicating” or “determining” means that the presence or absence of an allelic variant may be one of many factors that are considered when a subject's predisposition to a disease or disorder is evaluated. Thus a predisposition to a disease or disorder is not necessarily conclusively determined by only ascertaining the presence or absence of one or more allelic variants, but the presence of one of more of such variants is among an number of factors considered.

As used herein, “predisposition to develop a disease or disorder” means that a subject having a particular genotype and/or haplotype has a higher likelihood than one not having such a genotype and/or haplotype for developing a particular disease or disorder.

As used herein, “transgenic animal” refers to any animal, preferably a non-human animal, e.g. a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of a protein. However, transgenic animals in which the recombinant gene is silent are also contemplated, as for example, using the FLP or CRE recombinase dependent constructs. Moreover, “transgenic animal” also includes those recombinant animals in which gene disruption of one or more genes is caused by human intervention, including both recombination and antisense techniques.

As used herein, “associated” refers to coincidence with the development or manifestation of a disease, condition or phenotype. Association may be due to, but is not limited to, genes responsible for housekeeping functions, those that are part of a pathway that is involved in a specific disease, condition or phenotype and those that indirectly contribute to the manifestation of a disease, condition or phenotype.

As used herein, “high serum cholesterol” refers to a level of serum cholesterol that is greater than that considered to be in the normal range for a given age in a population, e.g., about 5.25 mmoles/L or greater, i.e., approximately one standard deviation or more away from the age-adjusted mean.

As used herein, “low serum HDL” refers to a level of serum HDL that is less than that considered to be in the normal range for a given age in a population, e.g. about 1.11 mmoles/L or less, i.e., approximately one standard deviation or more away from the age-adjusted mean.

As used herein, “cardiovascular disease” refers to any manifestation of or predisposition to cardiovascular disease including, but not limited to, coronary artery disease and myocardial infarction. Included in predisposition is the manifestation of risks factors such as high serum cholesterol levels and low serum HDL levels.

As used herein, “target nucleic acid” refers to a nucleic acid molecule which contains all or a portion of a polymorphic region of a gene of interest.

As used herein, “signal moiety” refers to any moiety that allows for the detection of a nucleic acid molecule. Included are moieties covalently attached to nucleic acids and those that are not.

As used herein, “biologically active agent that modulates serum cholesterol” refers to any drug, small molecule, nucleic acid (sense and antisense), protein, peptide, lipid, carbohydrate etc. or combination thereof, that exhibits some effect directly or indirectly on the cholesterol measured in a subject's serum.

As used herein, “biologically active agent that modulates serum HDL” refers to any drug, small molecule, nucleic acid (sense and antisense), protein, peptide, lipid, carbohydrate etc. or combination thereof that exhibits some effect directly or indirectly on the HDL measured in a subject's serum.

As used herein, “expression and/or activity” refers to the level of transcription or translation of the COX6B or GPI-1 gene, mRNA stability, protein stability or biological activity.

As used herein, “cardiovascular drug” refers to a drug used to treat cardiovascular disease or a risk factor for the disease, either prophylactically or after a risk factor or disease condition has developed. Cardiovascular drugs include those drugs used to lower serum cholesterol and those used to alter the level of serum HDL.

As used herein, “combining” refers to contacting the biologically active agent with a cell or animal such that the agent is introduced into the cell or animal. For a cell any method that results in an agent traversing the plasma membrane is useful. For an animal any of the standard routes of administration of an agent, e.g. oral, rectal, transmucosal, intestinal, intravenous, intraperitoneal, intraventricular, subcutaneous, intramuscular, etc., can be utilized.

As used herein,“positive response” refers to improving or ameliorating at least one symptom or detectable characteristic of a disease or condition, e.g., lowering serum cholesterol levels or raising serum HDL levels.

As used herein, “biological sample” refers to any cell type or tissue of a subject from which nucleic acid, particularly DNA, can be obtained.

As used herein, “array” refers to a collection of three or more items, such a collection of immobilized nucleic acid probes arranged on a solid substrate, such as silica, polymeric materials or glass.

As used herein, a composition refers to any mixture. It may be a solution, a suspension, liquid, powder, a paste, aqueous, non-aqueous or any combination thereof.

As used herein, a combination refers to any association between two or among more items.

As used herein, “kit” refers to a package that contains a combination, such as one or more primers or probes used to amplify or detect polymorphic regions of genes associated with cardiovascular disease, optionally including instructions and/or reagents for their use.

As used herein “specifically hybridizes” refers to hybridization of a probe or primer only to a target sequence preferentially to a non-target sequence. Those of skill in the art are familiar with parameters that affect hybridization; such as temperature, probe or primer length and composition, buffer composition and salt concentration and can readily adjust these parameters to achieve specific hybridization of a nucleic acid to a target sequence.

As used herein “nucleic acid” refers to polynucleotides such as deoxyribonucleic acid (DNA) and ribonucleic acid (RNA). The term should also be understood to include, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, “mass spectrometry” encompasses any suitable mass spectrometric format known to those of skill in the art. Such formats include, but are not limited to, Matrix-Assisted Laser Desorption/Ionization, Time-of-Flight (MALDI-TOF), Electrospray (ES), IR-MALDI (see, e.g., published International PCT Application No. 99/57318 and U.S. Pat. No. 5,118,937) Ion Cyclotron Resonance (ICR), Fourier Transform and combinations thereof. MALDI, particular UV and IR, are among the preferred formats.

B. Cytochrome c Oxidase VIb Gene

Cytochrome c oxidase (COX) is a mitochondrial enzyme complex integrated in the inner membrane. It transfers electrons from cytochrome to molecular oxygen in the terminal reaction of the respiratory chain in eukaryotic cells. COX contains of three large subunits encoded by the mitochondrial genome and 10 other subunits, encoded by nuclear genes. The three subunits encoded by mitochondrial genome are responsible for the catalytic activity. The cytochrome c oxidase subunit VIb (COX6B) is one of the nuclear gene products. The function of the nuclear encoded subunits is unknown. One proposed role is in the regulation of catalytic activity; specifically the rate of electron transport and stoichiometry of proton pumping. Other proposed roles are not directly related to electron transport and include energy-dependent calcium uptake and protein import by the mitochondrion. Proteolytic removal of subunits VIa and VIb has been associated with loss of calcium transport in reconstituted vesicles. Steady-state levels of the COX6B transcript are different in different tissues (Taanman et al., Gene (1990), 93:285).

The COX6B gene is generically used to include the human COX6B gene and its homologs from rat, mouse, guinea pig, etc.

Several single nucleotide polymorphism have been identified in the human COX6B gene. One of these is located at position 86 and is a C to T transversion which is manifested as a silent mutation in the coding region, ACC to ACT (threonine to threonine)(SEQ ID NO.: 2). Although this is a silent mutation at the amino acid level, it may represent an alteration that changes codon usage, or it may effect mRNA stability or it may be in linkage disequilibrium with a non-silent change. Other known single nucleotide polymorphisms of the COX6B gene include, but are not limited to, those listed in Table 1.

TABLE 1

| Gene | GenBank Accession No. | SNP | SNP Location |
|---|---|---|---|
| COX6B | NM_001863 | C/T | 86 |
| (SEQ ID NO.: 1) | | A/G | 60 |
| | | A/T | 324 |
| | | A/T | 123 |

Based on methods disclosed herein and those used in the art, one of skill would be able to utilize all the SNPs described and find additional polymorphic regions of the COX6B gene to determine whether allelic variants of these regions are associated with high cholesterol levels and cardiovascular disease.

C. GPI-1 Gene

Glycosylphosphatidylinositol (GPI) functions to anchor various eukaryotic proteins to membranes and is essential for their surface expression. Thus, a defect in GPI anchor synthesis affects various functions of cell, tissues and organs. Biosynthesis of glycosylphosphatidylinositol (GPI) is initiated by the transfer of N-acetylglucosamine (GIcNAc) from UDP-GIcNAc to phosphatidylinositol (PI) and is catalyzed by a GIcNAc transferase, GPI-GIcNAc transferase (GPI-GnT). Four mammalian gene products form a protein complex that is responsible for this enzyme activity (PIG-A, PIG-H, PIG-C and GPI-1). PIG-A, PIG-H, PIG-C are required for the first step in GPI anchor biosynthesis; GPI-1 is not. Stabilization of the enzyme complex, rather than participation in GIcNAc transfer, has been suggested as a possible role for GPI-1 (Watanabe et al. EMBO (1998) 17: 877).

The GPI-1 gene is generically used to include the human GPI-1 gene and its homologs from rat, mouse, guinea pig, etc.

A polymorphism has been identified at position 2577 of the human GPI-1 gene. This is a G to A transversion. This SNP is located in the 3′ untranslated region of the mRNA, and does not affect protein structure, but may affect mRNA stability or may be in linkage disequilibrium with a non-silent change. Other known single nucleotide polymorphisms of the GPI-1 gene include, but are not limited to, those listed in Table 2.

TABLE 2

| | GenBank | | |
|---|---|---|---|
| Gene | Accession No. | SNP | SNP Location |
| GPI-1 | NM_004204 | C/T | 2829 |
| (SEQ ID NOS.: 6, 7) | | A/G | 2577 |
| | | C/T | 2519 |
| | | C/T | 2289 |
| | | C/T | 1938 |
| | | C/G | 1563 |
| | | A/G/C/T | 2664 |
| | | A/G | 2656 |
| | | A/C/T | 2167 |
| | | G/C/A | 2166 |

Based on methods disclosed herein and those used in the art, one of skill would be able to use all the described SNPs and find additional polymorphic regions of the GPI-1 gene to determine whether allelic variants of these regions are associated with low levels of HDL and cardiovascular disease.

D. Other Genes and Polymorphism Associated with Cardiovascular Disease

Many other genes and polymorphisms contained within them have been associated with risks factors for cardiovascular disease (aberrations in lipid metabolism; specifically high levels of serum cholesterol and low levels of HDL, etc.) and/or the clinical phenotypes of atherosclerosis and cardiovascular disease. Table 3 presents a list of some of these genes and some associated polymorphisms (SNPs): cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase (LIPC); E-selectin; G protein beta 3 subunit and angiotensin II type 1 receptor gene. The SNP locations are based on the GenBank sequence. Table 3 is not meant to be exhaustive, as one of skill in the art based on the disclosure would be able to readily use other known polymorphisms in these and other genes, new polymorphisms discovered in previously identified genes and newly identified genes and polymorphisms in the methods and compositions disclosed herein.

TABLE 3

| | GenBank | | SNP |
|---|---|---|---|
| Gene | Accession No. | SNP | Location |
| CETP | NM_000078 | C/A | 991 |
| (SEQ ID NOS.: 11, 12) | | C/T | 196 |
| | | A/G | 1586 |
| | | A/G | 1394 |
| | | A/G | 1439 |
| | | C/G | 1297 |
| | | C/T | 766 |
| | | G/A | 1131 |
| | | G/A | 1696 |
| LPL | NM_000237 | A/G | 1127 |
| (SEQ ID NOS.: 13, 14) | | A/C | 3447 |
| | | C/T | 1973 |
| | | C/T | 3343 |
| | | G/A | 2851 |
| | | C/T | 3272 |
| | | A/T | 2428 |
| | | T/C | 2743 |
| | | G/A | 1453 |
| | | C/A | 3449 |
| | | G/A | 1282 |
| | | G/A | 579 |
| | | A/C | 1338 |
| | | A/G/T/C | 2416-2426 |
| | | A/G | 2427 |
| | | C/T | 1302 |
| | | G/A | 609 |
| | | G/C | 1595 |
| | | G/A | 1309 |
| | | C/T | 2454 |
| | | C/T | 2988 |
| | | G/A | 280 |
| | | G/A | 1036 |
| APO A4 | NM_000482 | G/T | 1122 |
| (SEQ ID NOS.: 15, 16) | | G/C | 1033 |
| | | G/A | 1002 |
| | | C/T | 960 |
| | | C/T | 894 |
| | | G/A | 554 |
| | | G/A | 950 |
| | | T/C | 336 |
| | | G/A | 334 |
| | | C/T | 330 |
| | | A/G | 201 |
| | | A/G | 16 |
| | | A/T | 1213 |
| APO E | NM_000041 | C/T | 448 |
| (SEQ ID NOS.: 17, 18) | | G/A | 448 |
| (mRNA) | | C/T | 586 |
| | | C/T | 197 |
| | | C/T | 540 |
| Hepatic Lipase | NM_000236 | C/G | 680 |
| (SEQ ID NOS.: 19, 20) | | G/A | 1374 |
| | | G/A | 701 |
| | | C/A | 1492 |
| | | A/G | 648 |
| | | G/C | 729 |
| | | G/A | 340 |
| | | G/T | 522 |
| PON 1 | NM_000446 | A/T | 172 |
| (SEQ ID NOS.: 21, 22) | | A/G | 584 |
| | | G/C | 190 |
| PON 2 | XM_004947 | C/G | 475 |
| (SEQ ID NOS.: 23, 24) | | C/G | 964 |
| APO C3 | NM_000040 | C/T | 148 |
| (SEQ ID NOS.: 25, 26) | | T/A | 471 |
| | | G/C | 386 |
| | | G/T | 417 |
| | | T/A | 495 |
| ABC 1 | XM_005567 | G/A | 8591 |
| (SEQ ID NOS.: 27, 28) | | | |
| APO A1 | NM_000039 | C/G | 770 |
| (SEQ ID NOS.: 29, 30) | | G/A | 656 |
| | | C/G | 589 |
| | | C/G | 414 |
| | | A/T | 430 |
| | | C/T | 708 |
| | | C/T | 221 |
| | | T/G | 223 |
| | | C/T | 597 |
| | | A/G | 340 |
| | | G/C | 690 |
| APO B | NM_000384 | A/G/C/T | 13141 |
| (SEQ ID NOS.: 31, 32) | | A/G/C/T | 12669 |
| | | C/T | 11323 |
| | | G/C | 10422 |
| | | A/C | 10408 |
| | | C/G | 10083 |
| | | C/T | 7064 |
| | | C/T | 6666 |
| | | C/T | 1980 |
| | | C/G | 5751 |
| | | C/T | 7673 |
| | | C/A/G/T | 8344 |
| | | G/C/T/A | 4393 |
| | | A/C/T/G | 5894 |
| | | A/T | 12019 |
| | | C/T | 11973 |
| | | G/C/T/A | 7065 |
| | | C/G | 947 |
| | | C/G | 7331 |
| | | A/G | 7221 |
| | | G/C | 6402 |
| | | G/C | 3780 |
| | | C/G | 1661 |
| | | A/T | 8167 |
| | | C/A | 8126 |
| | | C/T | 421 |
| | | C/T | 1981 |
| | | G/A | 12510 |
| | | G/C | 12937 |
| APO B (con't) | | G/A | 11042 |
| | | C/T | 2834 |
| | | A/G | 5869 |
| | | A/G | 11962 |
| | | C/G | 4439 |
| | | G/A | 7824 |
| | | G/A | 13569 |
| | | G/A | 9489 |
| | | G/A | 2325 |
| | | G/A | 10259 |
| | | C/G | 14 |
| MTHFR | NM_005957 | G/A | 5442 |
| (SEQ ID NOS.: 33, 34) | | A/G | 5113 |
| | | A/G | 5113 |
| | | A/G | 5110 |
| | | A/G | 5102 |
| | | A/C/T | 5097 |
| | | A/C/T | 5097 |
| | | C/T | 5079 |
| | | C/T | 5079 |
| | | T/C | 5071 |
| | | T/C | 5071 |
| | | T/C | 5051 |
| | | G/A | 5012 |
| | | C/A | 5000 |
| | | A/G | 4998 |
| | | A/G | 4994 |
| | | A/G | 4994 |
| | | A/G | 4994 |
| | | C/T | 4991 |
| | | C/T | 4991 |
| | | C/T | 4991 |
| | | A/G | 4986 |
| | | A/G | 4986 |
| | | A/G | 4986 |
| | | C/T | 4985 |
| | | T/A | 4982 |
| | | T/G | 4981 |
| | | T/C | 4981 |
| | | T/C | 4981 |
| MTHFR (con't) | | G/C/A | 4967 |
| | | G/A | 4963 |
| | | A/G | 4962 |
| | | G/C/T | 4962 |
| | | A/C/G/T | 4961 |
| | | A/C/T | 4961 |
| | | A/C | 4961 |
| | | A/C | 4961 |
| | | A/C/T | 4960 |
| | | T/C | 4938 |
| | | T/C | 4937 |
| | | T/C | 4933 |
| | | G/C/T | 4933 |
| | | C/T | 4929 |
| | | C/T | 4929 |
| | | T/A/G | 4929 |
| | | A/G | 4928 |
| | | G/C | 4928 |
| | | C/G | 4927 |
| | | G/A | 4923 |
| | | C/T | 4919 |
| | | A/T/G | 4913 |
| | | C/T | 4912 |
| | | A/T | 4903 |
| | | C/T | 4902 |
| | | A/G | 4900 |
| | | G/A | 4898 |
| | | G/T | 4898 |
| | | C/T | 4897 |
| | | G/T | 4894 |
| | | T/C/G | 4836 |
| | | C/T | 3862 |
| | | C/T | 4922 |
| | | C/T | 4959 |
| | | T/C | 4981 |
| | | A/G | 4994 |
| | | A/G | 5044 |
| | | T/C | 5051 |
| | | G/C | 5066 |
| | | C/T | 5079 |
| MTHFR (con't) | | C/A/G | 5085 |
| | | C/T | 5092 |
| | | A/G | 5103 |
| | | A/G | 5113 |
| | | C/T | 1021 |
| E-Selectin | NM_000450 | G/A | 3484 |
| (SEQ ID NOS.: 35, 36) | | G/A | 3093 |
| | | T/G | 2939 |
| | | T/C | 2902 |
| | | C/T | 1937 |
| | | C/T | 1916 |
| | | C/T | 1839 |
| | | C/T | 1805 |
| | | C/T | 1518 |
| | | G/C | 1377 |
| | | C/T | 1376 |
| | | G/A | 999 |
| | | T/C | 857 |
| | | A/C | 561 |
| | | C/G | 506 |
| | | A/G | 392 |
| | | G/T | 98 |
| G protein β3 subunit | NM_002075 | C/T | 1828 |
| (SEQ ID NOS.: 37, 38) | | C/T | 1546 |
| | | G/T | 1431 |
| | | G/A | 1231 |
| | | C/T | 1230 |
| Angiotensin II type 1 | NM_00686 | G/A | 1453 |
| receptor gene | | C/G | 968 |
| (SEQ ID NOS.: 39, 40) | | G/C | 966 |
| | | T/C | 941 |
| | | G/A | 894 |
| | | T/C | 659 |

Assays to identify the nucleotide present at the polymorphic site include those described herein and all others known to those who practice the art.

For some of the SNPs described above, there are provided a description of the MassEXTEND™ reaction components that can be utilized to determine the allelic variant that is present. Included are the forward and reverse primers used for amplification. Also included are the MassEXTEND™ primer used in the primer extension reaction and the extended MassEXTEND™ primers for each allele. MassEXTEND™ reactions are carried out and the products analyzed as described in Examples 2 and 3.

CETP

| Position 991 (C/A) | | |
|---|---|---|
| PCR primers: | | |
| Forward: | ACTGCCTGATAACCATGCTG | (SEQ ID NO.: 41) |
| Reverse: | ATACTTACACACCAGGAGGG | (SEQ ID NO.: 42) |
| MassEXTENDTM Primer: | ATGCCTGCTCCAAAGGCAC | (SEQ ID NO.: 43) |
| Primer Mass: | 5757.8 | |
| Extended Primer-Allele C: | ATGCCTGCTCCAAAGGCACC | (SEQ ID NO.: 44) |
| Extended Primer Mass: | 6030.9 | |
| Extended Primer-Allele A: | ATGCCTGCTCCAAAGGCACAT | (SEQ ID NO.: 45) |
| Extended Primer Mass: | 6359.2 | |
| Position 196 (CIT) | | |
| PCR primers: | | |
| Forward: | TACTTCTGGTTCTCTGAGCG | (SEQ ID NO.: 46) |
| Reverse: | ACTCACCTTGAACTCGTCTC | (SEQ ID NO.: 47) |
| MassEXTEND ™ Primer: | TGGTTCTCTGAGCGAGTCTT | (SEQ ID NO.: 48) |
| Primer Mass: | 6130 | |
| Extended Primer-Allele C: | TGGTTCTCTGAGCGAGTCTTC | (SEQ ID NO.: 49) |
| Extended Primer Mass: | 6707.4 | |
| Extended Primer-Allele T: | TGGTTCTCTGAGCGAGTCTTTC | (SEQ ID NO.: 50) |
| Extended Primer Mass: | 6333.1 | |
| Position 1586 (AIG) | | |
| POR primers: | | |
| Forward: | TGCAGATGGACTTTGGCTTC | (SEQ ID NO.: 51) |
| Reverse: | TGCTTGCCTTCTGCTACAAG | (SEQ ID NO.: 52) |
| MassEXTENDTM Primer: | CTTCCCTGAGCACCTGCTG | (SEQ ID NO.: 53) |
| Primer Mass: | 5715.7 | |
| Extended Primer-Allele G: | CTTCCCTGAGCACCTGCTGGT | (SEQ ID NO.: 54) |
| Extended Primer Mass: | 6333.1 | |
| Extended Primer-Allele A: | CTTCCCTGAGCACCTGCTGA | (SEQ ID NO.: 55) |
| Extended Primer Mass: | 601 2.9 | |
| APOA4 | | |
| Position 1122 (GIT) | | |
| POR primers: | | |
| Forward: | AACAGCTCAGGACGAAACTG | (SEQ ID NO.: 56) |
| Reverse: | AGAAGGAGTTGACCTTGTCC | (SEQ ID NO.: 57) |
| MassEXTEND ™ Primer: | GGAAGCTCAAGTGGCCTTC | (SEQ ID NO.: 5)8) |
| Primer Mass: | 5828.8 | |
| Extended Primer-Allele G: | GGAAGCTCAAGTGGCCTTCC | (SEQ ID NO.: 59) |
| Extended Primer Mass: | 6102.0 | |
| Extended Primer-Allele T: | GGAAGCTCAAGTGGCCTTCAAC | (SEQ ID NO.: 60) |
| Extended Primer Mass: | 6728.4 | |
| Position 1033 (GIC) | | |
| PCR primers: | | |
| Forward: | AAGTCACTGGCAGAGCTGG | (SEQ ID NO.: 61) |
| Reverse: | GCACCAGGGCTTTGTTGAAG | (SEQ ID NO.: 62) |
| MassEXTEND ™ Primer: | TTTTCCCCGTAGGGCTCCA | (SEQ ID NO.: 63) |
| Primer Mass: | 5730.7 | |
| Extended Primer-Allele G: | TTTTCCCCGTAGGGCTCCAC | (SEQ ID NO.: 64) |
| Extended Primer Mass: | 6003.9 | |
| Extended Primer-Allele C: | TTTTCCCCGTAGGGCTCCAGC | (SEQ ID NO.: 65) |
| Extended Primer Mass: | 6333.1 | |
| Position 1002 (G/A) | | |
| PCR primers: | | |
| Forward: | TGCAGAAGTCACTGGCAGAG | (SEQ ID NO.: 66) |
| Reverse: | GTTGAAGTTTTCCCCGTAGG | (SEQ ID NO.: 67) |
| MassEXTEND ™ Primer: | ACTCCTCCACCTGCTGGTC | (SEQ ID NO.: 68) |
| Primer Mass: | 5675.7 | |
| Extended Primer-Allele G: | ACTCCTCCACCTGCTGGTCC | (SEQ ID NO.: 69) |
| Extended Primer Mass: | 5948.9 | |
| Extended Primer-Allele A: | ACTCCTCCACCTGCTGGTCTA | (SEQ ID NO.: 70) |
| Extended Primer Mass: | 6277.1 | |
| Position 960 (CIT) | | |
| PCR primers: | | |
| Forward: | AGGACGTGCGTGGCAACCTG | (SEQ ID NO.: 71) |
| Reverse: | AGCTGTGCCAGTGACTTCTG | (SEQ ID NO.: 72) |
| MassEXTEND ™ Primer: | GTGACTTCTGCAGCCCCTC | (SEQ ID NO.: 73) |
| Primer Mass: | 571 5.7 | |
| Extended Primer-Allele T: | GTGACTTCTGCAGCCCCTCA | (SEQ ID NO.: 74) |
| Extended Primer Mass: | 601 2.9 | |
| Extended Primer-Allele C: | GTGACTTCTGGAGCCCCTCGGT | (SEQ ID NO.: 75) |
| Extended Primer Mass: | 6662.3 | |
| Position 894 (CIT) | | |
| PCR primers: | | |
| Forward: | CCTGACCTTCCAGATGAAG | (SEQ ID NO.: 76) |
| Reverse: | TCAGGTTGCCACGCACGTC | (SEQ ID NO.: 77) |
| MassEXTEND ™ Primer: | CAGGATCTCGGCCAGTGC | (SEQ ID NO.: 78) |
| Primer Mass: | 5500.6 | |
| Extended Primer-Allele C: | CAGGATCTCGGCCAGTGCC | (SEQ ID NO.: 79) |
| Extended Primer Mass: | 5773.8 | |
| Extended Primer-Allele T: | CAGGATCTCGGCCAGTGCTG | (SEQ ID NO.: 80) |
| Extended Primer Mass: | 61 18.0 | |
| Position 554 (G/A) | | |
| PCR primers: | | |
| Forward: | ACCTGCGAGAGCTTCAGCAG | (SEQ ID NO.: 81) |
| Reverse: | TCTCCATGCGCTGTGCGTAG | (SEQ ID NO.: 82) |
| MassEXTEND ™ Primer: | AGCTGCGCACCCAGGTCA | (SEQ ID NO.: 83) |
| Primer Mass: | 5469.6 | |
| Extended Primer-Allele A: | AGCTGCGCACCCAGGTCAA | (SEQ ID NO.: 84) |
| Extended Primer Mass: | 5766.8 | |
| Extended Primer-Allele G: | AGCTGCGCACCCAGGTCAGC | (SEQ ID NO.: 85) |
| Extended Primer Mass: | 6072.0 | |
| APOE | | |
| Position 448 (CIT) | | |
| PCR primers: | | |
| Forward: | TGTCCAAGGAGCTGCAGGC | (SEQ ID NO.: 86) |
| Reverse: | CTTACGCAGCTTGCGCAGGT | (SEQ ID NO.: 87) |
| MassEXTEND ™ Primer: | GCGGAGATGGAGGACGTG | (SEQ ID NO.: 88) |
| Primer Mass: | 5629.7 | |
| Extended Primer-Allele C: | GCGGACATGGAGGACGTGC | (SEQ ID NO.: 89) |
| Extended Primer Mass: | 5902.8 | |
| Extended Primer-Allele T: | GCGGACATGGAGGACGTGTG | (SEQ ID NO.: 90) |
| Extended Primer Mass: | 6247.1 | |
| LPL | | |
| Position 1127 (A/G) | | |
| PCR primers: | | |
| Forward: | GTTGTAGAAAGAACCGCTGC | (SEQ ID NO.: 91) |
| Reverse: | GAGAACGAGTCTTCAGGTAC | (SEQ ID NO.: 92) |
| MassEXTEND ™ Primer: | ACAATCTGGGCTATGAGATCA | (SEQ ID NO.: 93) |
| Primer Mass: | 6454.2 | |
| Extended Primer-Allele A: | ACAATCTGGGCTATGAGATCAA | (SEQ ID NO.: 94) |
| Extended Primer Mass: | 6751 .4 | |
| Extended Primer-Allele G: | ACAATCTGGGCTATGAGATCAGT | (SEQ ID NO.: 95) |
| Extended Primer Mass: | 7071 .6 | |
| Position 3447 (A/C) | | |
| PCR primers: | | |
| Forward: | GACTCTACACTGCATGTCTC | (SEQ ID NO.: 96) |
| Reverse: | ACCCTTCTGAAAAGGAGAGG | (SEQ ID NO.: 97) |
| MassEXTENDTM Primer: | GAGGAGAGACAAGGCAGATA | (SEQ ID NO.: 98) |
| Primer Mass: | 6273.1 | |
| Extended Primer-Allele A: | GAGGAGAGACAAGGCAGATAT | (SEQ ID NO.: 99) |
| Extended Primer Mass: | 6561.3 | |
| Extended Primer-Allele C: | GAGGAGAGACAAGGCAGATAGT | (SEQ ID NO.: 100) |
| Extended Primer Mass: | 6890.5 | |
| Position 1973 (C/TI | | |
| PCR primers: | | |
| Forward: | AAAGGTTCAGTTGCTGCTGC | (SEQ ID NO.: 101) |
| Reverse: | GCTGGGGAAGGTCTAATAAC | (SEQ ID NO.: 102) |
| MassEXTENDTM Primer: | GTTGCTGCTGCCTCGAATG | (SEQ ID NO.: 103) |
| Primer Mass: | 5770.7 | |
| Extended Primer-Allele C: | GTTGCTGCTGCCTCGAATCC | (SEQ ID NO.: 104) |
| Extended Primer Mass: | 6043.9 | |
| Extended Primer-Allele T: | GTTGCTGCTGCCTCGAATCTG | (SEQ ID NO.: 105) |
| Extended Primer Mass: | 6388.2 | |
| LIPC | | |
| Position 680 (CIG) | | |
| PCR primers: | | |
| Forward: | CGTCTTTCTCCAGATGATGC | (SEQ ID NO.: 106) |
| Reverse: | AGTGTCCTATGGGCTGTTTG | (SEQ ID NO.: 107) |
| MassEXTEND ™ Primer: | GGATGCCATTCATACCTTTAC | (SEQ ID NO.: 108) |
| Primer Mass: | 6556.1 | |
| Extended Primer-Allele C: | GGATGCCATTCATACCTTTACC | (SEQ ID NO.: 109) |
| Extended Primer Mass: | 6629.3 | |
| Extended Primer-Allele G: | GGATGCCATTCATACCTTTACGC | (SEQ ID NO.: 110) |
| Extended Primer Mass: | 6958.5 | |
| Position 1374 (GIA) | | |
| PCR primers: | | |
| Forward: | TGGGAAAACAGTGCAGTGTG | (SEQ ID NO.: 111) |
| Reverse: | TGATCGTCTTCAGAACGAGG | (SEQ ID NO.: 112) |
| MassEXTEND ™ Primer: | CCAGACCATCATCCCATGGA | (SEQ ID NO.: 113) |
| Primer Mass: | 6030.9 | |
| Extended Primer-Allele A: | CCAGACCATCATCCCATGGAA | (SEQ ID NO.: 114) |
| Extended Primer Mass: | 6328.1 | |
| Extended Primer-Allele G: | CCAGACCATCATCCCATGGAGC | (SEQ ID NO.: 115) |
| Extended Primer Mass: | 6633.3 | |
| Position 701 (G/A) | | |
| PCR primers: | | |
| Forward: | CAGCAATCGTCTTTCTCCAG | (SEQ ID NO.: 116) |
| Reverse: | TCCTATGGGCTGTTTGATGC | (SEQ ID NO.: 117) |
| MassEXTEND ™ Primer: | GTCTTTCTCCAGATGATGCCA | (SEQ ID NO.: 118) |
| Primer Mass: | 6372.2 | |
| Extended Primer-Allele A: | GTCTTTCTCCAGATGATGCCAA | (SEQ ID NO.: 119) |
| Extended Primer Mass: | 6669.4 | |
| Extended Primer-Allele G: | GTCTTTCTCCAGATGATGCCAGT | (SEQ ID NO.: 120) |
| Extended Primer Mass: | 6989.6 | |

E. Databases

Databases for determining an association between polymorphic regions of genes and intermediate and clinical phenotypes, comprise biological samples (e.g., blood) which provide a source of nucleic acid and clinical data covering diseases (e.g., age, sex, ethnicity medical history and family medical history) from both individuals exhibiting the phenotype (intermediate phenotype (risk factor) or clinical phenotype (disease)) and those who do not. These databases include human population groups such as twins, diverse affected families, isolated founder populations and drug trial subjects. The quality and consistency of the clinical resources are of primary importance.

F. Association Studies

The examples set forth below utilized an extreme trait analysis to discover an association between an allelic variant of the COX6B gene and high cholesterol and an association between an allelic variant of the GPI-1 gene and low HDL. This analysis is based on comparing a pair of pools of DNA from individuals who exhibit respectively hypo or hypernormal levels of a biochemical trait (e.g., cholesterol or HDL) and individually examining SNPs for a difference in allelic frequency between the pools. An association is considered to be positive if a statistically significant value of at least 3.841 using a 1-degree-of-freedom chi-squared test of association, p=0.05, is obtained. Standard multiple testing corrections are applied if more than one SNP is considered at a time, i.e., multiple SNPs are tested during the same study. Although not always required, it may be necessary to further examine the frequency of allelic variants in other populations, including those exhibiting normal levels of the given trait.

For a qualitative trait (e.g., hypertension) association studies are based on determining the occurrence of certain alleles in a given population of diseased vs. healthy individuals.

Allelic variants of COX6B, GPI-1 and other genes found to associate with high cholesterol, low HDL and/or cardiovascular disease can represent useful markers for indicating a predisposition for developing a risk factor for cardiovascular disease. These allelic variants may not necessarily represent functional variants affecting the expression, stability, or activity of the encoded protein product. Those of skill in the art would be able to determine which allelic variants are to be used, alone or in conjunction with other variants, only for indicating a predisposition for cardiovascular disease or for profiling of drug reactivity and for determining those which may be also useful for screening for potential therapeutics.

Any method used to determine association can be utilized to discover or confirm the association of other polymorphic regions in the COX6B gene, the GPI-1 gene or any other gene that may be associated with cardiovascular disease.

G. Detection of Polymorphisms

1. Nucleic Acid Detection Method

Generally, these methods are based in sequence-specific polynucleotides, oligonucleotides, probes and primers. Any method known to those of skill in the art for detecting a specific nucleotide within a nucleic acid sequence or for determining the identity of a specific nucleotide in a nucleic acid sequence is applicable to the methods of determining the presence or absence of an allelic variant of a COX6B gene or GPI-1 gene or another gene associated with cardiovascular disease. Such methods include, but are not limited to, techniques utilizing nucleic acid hybridization of sequence-specific probes, nucleic acid sequencing, selective amplification, analysis of restriction enzyme digests of the nucleic acid, cleavage of mismatched heteroduplexes of nucleic acid and probe, alterations of electrophoretic mobility, primer specific extension, oligonucleotide ligation assay and single-stranded conformation polymorphism analysis. In particular, primer extension reactions that specifically terminate by incorporating a dideoxynucleotide are useful for detection. Several such general nucleic acid detection assays are described in U.S. Pat. No. 6,030,778.

a. Primer Extension-Based Methods

Several primer extension-based methods for determining the identity of a particular nucleotide in a nucleic acid sequence have been reported (see, e.g., PCT Application No. PCT/US96/03651 (WO96/29431), PCT Application No. PCT/US97/20444 (WO 98/20019), PCT Application No. PCT/US91/00046 (WO91/13075), and U.S. Pat. No. 5,856,092). In general, a primer is prepared that specifically hybridizes adjacent to a polymorphic site in a particular nucleic acid sequence. The primer is then extended in the presence of one or more dideoxynucleotides, typically with at least one of the dideoxynucleotides being the complement of the nucleotide that is polymorphic at the site. The primer and/or the dideoxynucleotides may be labeled to facilitate a determination of primer extension and identity of the extended nucleotide.

In a preferred method, primer extension and/or the identity of the extended nucleotide(s) are determined by mass spectrometry (see, e.g., PCT Application Nos. PCT/US96/03651 (WO96/29431) and PCT/US97/20444 (WO 98/20019)).

b. Polymorphism-Specific Probe Hybridization

A preferred detection method is allele specific hybridization using probes overlapping the polymorphic site and having about 5, 10, 15, 20, 25, or 30 nucleotides around the polymorphic region. The probes can contain naturally occurring or modified nucleotides (see U.S. Pat. No. 6,156,501). For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324: 163; Saiki et al. (1989) Proc. Natl Acad. Sci USA 86: 6230; and Wallace et al. (1979) Nucl. Acids Res. 6: 3543). Such allele specific oligonucleotide hybridization techniques may be used for the simultaneous detection of several nucleotide changes in different polymorphic regions. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid. In a preferred embodiment, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix, Santa Clara, Calif.). Mutation detection analysis using these chips comprising oligonucleotides, also termed “DNA probe arrays” is described e.g., in Cronin et al. (1996) Human Mutation 7: 244 and in Kozal et al. (1996) Nature Medicine 2: 753. In one embodiment, a chip includes all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

C. Nucleic Acid Amplification-Based Methods

In other detection methods, it is necessary to first amplify at least a portion of a COX6B gene, GPI-1 gene or another gene associated with cardiovascular disease prior to identifying the allelic variant. Amplification can be performed, e.g., by PCR and/or LCR, according to methods known in the art. In one embodiment, genomic DNA of a cell is exposed to two PCR primers and amplification is performed for a number of cycles sufficient to produce the required amount of amplified DNA. In preferred embodiments, the primers are located between 1 50 and 350 base pairs apart.

Alternative amplification methods include: self sustained sequence replication (Guatelli, J. C. et al. (1990) Proc. Natl. Acad. Sci. U.S.A. 87: 1874-1878); transcriptional amplification system (Kwoh, D. Y. et al. (1989) Proc. Natl. Acad. Sci. U.S.A. 86: 1173-1177), Q-Beta Replicase (Lizardi, P. M. et al. (1988) Bio/Technology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

Alternatively, allele specific amplification technology, which depends on selective PCR amplification may be used in conjunction with the alleles provided herein. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). In addition it may be desirable to introduce a restriction site in the region of the mutation to create cleavage-based detection (Gasparini et al. (1992) Mol. Cell Probes 6:1).

d. Nucleic Acid Sequencing-Based Methods

In one embodiment, any of a variety of sequencing reactions known in the art can be used to directly sequence at least a portion of the COX6B gene, GPI-1 gene or other gene associated with cardiovascular disease and to detect allelic variants, e.g., mutations, by comparing the sequence of the sample sequence with the corresponding wild-type (control) sequence. Exemplary sequencing reactions include those based on techniques developed by Maxam and Gilbert (Proc. Natl. Acad. Sci. USA (1977) 74:560) or Sanger (Sanger et al. (1977) Proc. Natl. Acad. Sci 74:5463). It is also contemplated that any of a variety of automated sequencing procedures may be used when performing the subject assays (Biotechniques (1995) 19:448), including sequencing by mass spectrometry (see, for example, U.S. Pat. No. 5,547,835 and International PCT Application No. WO 94/16101, entitled DNA Sequencing by Mass Spectrometry by H. Koster; U.S. Pat. No. 5,547,835 and International PCT Application No. WO 94/21822, entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track sequencing or an equivalent, e.g., where only one nucleotide is detected, can be carried out. Other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

e. Restriction Enzyme Digest Analysis

In some cases, the presence of a specific allele in nucleic acid, particularly DNA, from a subject can be shown by restriction enzyme analysis. For example, a specific nucleotide polymorphism can result in a nucleotide sequence containing a restriction site which is absent from the nucleotide sequence of another allelic variant.

f. Mismatch Cleavage

Protection from cleavage agents, such as, but not limited to, a nuclease, hydroxylamine or osmium tetroxide and with piperidine, can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g, RNA or DNA, obtained from a tissue sample. The double-stranded duplexes are treated with an agent, which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they differ (see, for example, Cotton et al. (1988) Proc. Natl Acad Sci USA 85: 4397; Saleeba et al. (1992) Methods Enzymol. 217: 286-295). The control or sample nucleic acid is labeled for detection.

g. Electrophoretic Mobility Alterations

In other embodiments, alteration in electrophoretic mobility is used to identify the type of allelic variant in the COX6B gene, GPI-1 gene or other gene associated with cardiovascular disease. For example, single-strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

h. Polyacrylamide Gel Electrophoresis

In yet another embodiment, the identity of an allelic variant of a polymorphic region in the COX6B gene, GPI-1 gene or other gene associated with cardiovascular disease is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

i. Oligonucleotide Ligation Assay (OLA)

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′ -amino group and a 5′-phosphorylated oligonucleotide to form a conjugate having a phosphoramidate linkage. In another variation of OLA described in Tobe et al. (1996) Nucl. Acids Res. 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

j. SNP Detection Methods

Also provided are methods for detecting single nucleotide polymorphisms. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment, a solution-based method for determining the identity of the nucleotide of a polymorphic site is employed (Cohen, D. et al. (French Patent 2,650,840; PCT Application No. WO91/02087)). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

k. Genetic Bit Analysis

An alternative method, known as Genetic Bit Analysis or GBA™ is described by Goelet, et al. (U.S. Pat. No. 6,004,744, PCT Application No. 92/15712). The method of Goelet, et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO91/02087), the method of Goelet, et al. is preferably a heterogeneous phase assay, in which the primer or the target molecule is immobilized to a solid phase.

I. Other Primer-Guided Nucleotide Incorporation Procedures

Other primer-guided nucleotide incorporation procedures for assaying polymorphic sites in DNA have been described (Komher, J. S. et al., Nucl. Acids Res. 17:7779-7784 (1989); Sokolov, B. P., Nucl. Acids Res. 18:3671 (1990); Syvanen, A. C., et al., Genomics 8:684-692 (1990), Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., Amer. J. Hum. Genet. 52:46-59 (1993)).

For determining the identity of the allelic variant of a polymorphic region located in the coding region of a gene, yet other methods than those described above can be used. For example, identification of an allelic variant which encodes a mutated protein can be performed by using an antibody specifically recognizing the mutant protein in, e.g., immunohistochemistry or immunoprecipitation. Binding assays are known in the art and involve, e.g., obtaining cells from a subject, and performing binding experiments with a labeled lipid, to determine whether binding to the mutated form of the protein differs from binding to the wild-type protein.

m. Molecular Structure Determination

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and SSCP.

n. Mass Spectrometric Methods

Nucleic acids can also be analyzed by detection methods and protocols, particularly those that rely on mass spectrometry (see, e.g., U.S. Pat. No. 5,605,798, allowed co-pending U.S. application Ser. No. 08/617,256, allowed co-pending U.S. application Ser. No. 08/744,481, U.S. application Ser. No. 08/990,851, International PCT Application No. WO 98/20019). These methods can be automated (see, e.g., co-pending U.S. application Ser. No. 09/285,481, which describes an automated process line). Preferred among the methods of analysis herein are those involving the primer oligo base extension (PROBE) reaction with mass spectrometry for detection (described herein and elsewhere, see e.g., U.S. application Ser. Nos. 08/617,256, 09/287,681, 09/287,682, 09/287,141 and 09/287,679, allowed co-pending U.S. application Ser. No. 08/744,481, International PCT Application No. PCT/US97/20444, published as International PCT Application No. WO 98/20019, and based upon U.S. application Ser. Nos. 08/744,481, 08/744,590, 08/746,036, 08/746,055, 08/786,988, 08/787,639, 08/933,792, 08/746,055, 08/786,988 and 08/787,639; see, also U.S. application Ser. No. 09/074,936, allowed U.S. application Ser. No. 08/787,639, and U.S. application Ser. Nos. 08/746,055 and 08/786,988, and published International PCT Application No. WO 98/20020).

A preferred format for performing the analyses is a chip based format in which the biopolymer is linked to a solid support, such as a silicon or silicon-coated substrate, preferably in the form of an array. More preferably, when analyses are performed using mass spectrometry, particularly MALDI, nanoliter volumes of sample are loaded on, such that the resulting spot is about, or smaller than, the size of the laser spot. It has been found that when this is achieved, the results from the mass spectrometric analysis are quantitative. The area under the peaks in the resulting mass spectra are proportional to concentration (when normalized and corrected for background). Methods for preparing and using such chips are described in allowed co-pending U.S. application Ser. No. 08/787,639, co-pending U.S. application Ser. Nos. 08/786,988, 09/364,774, 09/371,150 and 09/297,575; see, also U.S. application Ser. No. PCT/US97/20195, which published as International PCT Application No. WO 98/20020. Chips and kits for performing these analyses are commercially available from SEQUENOM under the trademark MassARRAY™. MassARRAY™ relies on the fidelity of the enzymatic primer extension reactions combined with the miniaturized array and MALDI-TOF (Matrix-Assisted Laser Desorption Ionization-Time of Flight) mass spectrometry to deliver results rapidly. It accurately distinguishes single base changes in the size of DNA fragments relating to genetic variants without tags.

Multiplex methods allow for the simultaneous detection of more than one polymorphic region in a particular gene or polymorphic regions in several genes. This is the preferred method for carrying out haplotype analysis of allelic variants of the COX6B and/or GPI-1 genes separately, or along with allelic variants of one or more other genes associated with cardiovascular disease.

Multiplexing can be achieved by several different methodologies. For example, several mutations can be simultaneously detected on one target sequence by employing corresponding detector (probe) molecules (e.g., oligonucleotides or oligonucleotide mimetics). The molecular weight differences between the detector oligonucleotides must be large enough so that simultaneous detection (multiplexing) is possible. This can be achieved either by the sequence itself (composition or length) or by the introduction of mass-modifying functionalities into the detector oligonucleotides (see below).

Mass modifying moieties can be attached, for instance, to either the 5′-end of the oligonucleotide, to the nucleobase (or bases), to the phosphate backbone, and to the 2′-position of the nucleoside (nucleosides) and/or to the terminal 3′-position. Examples of mass modifying moieties include, for example, a halogen, an azido, or of the type, XR, wherein X is a linking group and R is a mass-modifying functionality. The mass-modifying functionality can thus be used to introduce defined mass increments into the oligonucleotide molecule.

The mass-modifying functionality can be located at different positions within the nucleotide moiety (see, e.g., U.S. Pat. No. 5,547,835 and International PCT Application No. WO 94/21822). For example, the mass-modifying moiety, M, can be attached either to the nucleobase, (in case of the c⁷-deazanucleosides also to C-7), to the triphosphate group at the alpha phosphate or to the 2′-position of the sugar ring of the nucleoside triphosphate. Modifications introduced at the phosphodiester bond, such as with alpha-thio nucleoside triphosphates, have the advantage that these modifications do not interfere with accurate Watson-Crick base-pairing and additionally allow for the one-step post-synthetic site-specific modification of the complete nucleic acid molecule e.g., via alkylation reactions (see, e.g., Nakamaye et al. (1988) Nucl. Acids Res. 16:9947-59). Particularly preferred mass-modifying functionalities are boron-modified nucleic acids since they are better incorporated into nucleic acids by polymerases (see, e.g., Porter et al. (1995) Biochemistry 34:11963-11969; Hasan et al. (1996) Nucleic Acids Res. 24:2150-2157; Li et al. (1995) Nucl. Acids Res. 23:4495-4501).

Furthermore, the mass-modifying functionality can be added so as to affect chain termination, such as by attaching it to the 3′-position of the sugar ring in the nucleoside triphosphate. For those skilled in the art, it is clear that many combinations can be used in the methods provided herein. In the same way, those skilled in the art will recognize that chain-elongating nucleoside triphosphates can also be mass-modified in a similar fashion with numerous variations and combinations in functionality and attachment positions.

For example, without being bound to any particular theory, the mass-modification can be introduced for X in XR as well as using oligo-/polyethylene glycol derivatives for R. The mass-modifying increment (m) in this case is 44, i.e. five different mass-modified species can be generated by just changing m from 0 to 4 thus adding mass units of 45 (m=0), 89 (m=1), 133 (m=2), 177 (m=3) and 221 (m=4) to the nucleic acid molecule (e.g., detector oligonucleotide (D) or the nucleoside triphosphates, respectively). The oligo/polyethylene glycols can also be monoalkylated by a lower alkyl such as, but are not limited to, methyl, ethyl, propyl, isopropyl and t-butyl. Other chemistries can be used in the mass-modified compounds (see, e.g., those described in Oligonucleotides and Analogues, A Practical Approach, F. Eckstein, editor, IRL Press, Oxford, 1991).

In yet another embodiment, various mass-modifying functionalities, R, other than oligo/polyethylene glycols, can be selected and attached via appropriate linking chemistries, X. A simple mass-modification can be achieved by substituting H for halogens, such as F, Cl, Br and/or I, or pseudohalogens such as CN, SCN, NCS, or by using different alkyl, aryl or aralkyl moieties such as methyl, ethyl, propyl, isopropyl, t-butyl, hexyl, phenyl, substituted phenyl, benzyl, or functional groups such as CH₂F, CHF₂, CF₃, Si(CH₃)₃, Si(CH₃)₂(C₂H₅), Si(CH₃)(C₂H₅)₂, Si(C₂H₅)₃. Yet another mass-modification can be obtained by attaching homo- or heteropeptides through the nucleic acid molecule (e.g., detector (D)) or nucleoside triphosphates). One example, useful in generating mass-modified species with a mass increment of 57, is the attachment of oligoglycines (m) to nucleic acid molecules (r), e.g., mass-modifications of 74 (r=1, m=0), 131 (r=1, m=1), 188 (r=1, m=2), 245 (r=1, m=3) are achieved. Simple oligoamides also can be used, e.g., mass-modifications of 74 (r=1, m=0), 88 (r=2, m=0), 102 (r=3, m=0), 116(r=4, m=0), etc. are obtainable. Variations in additions to those set forth herein will be apparent to the skilled artisan.

Different mass-modified detector oligonucleotides can be used to simultaneously detect all possible variants/mutants simultaneously. Alternatively, all four base permutations at the site of a mutation can be detected by designing and positioning a detector oligonucleotide, so that it serves as a primer for a DNA/RNA polymerase with varying combinations of elongating and terminating nucleoside triphosphates. For example, mass modifications also can be incorporated during the amplification process.

A different multiplex detection format is one in which differentiation is accomplished by employing different specific capture sequences which are position-specifically immobilized on a flat surface (e.g., a ‘chip array’). If different target sequences T1-Tn are present, their target capture sites TCS1-TCSn will specifically interact with complementary immobilized capture sequences C1-Cn. Detection is achieved by employing appropriately mass differentiated detector oligonucleotides D1 -Dn, which are mass modifying functionalities M1-Mn.

o. Other Methods p Additional methods of analyzing nucleic acids include amplification-based methods including polymerase chain reaction (PCR), ligase chain reaction (LCR), mini-PCR, rolling circle amplification, autocatalytic methods, such as those using OJ replicase, TAS, 3SR, and any other suitable method known to those of skill in the art.

Other methods for analysis and identification and detection of polymorphisms, include but are not limited to, allele specific probes, Southern analyses, and other such analyses.

2. Primers and Probes

Primers refer to nucleic acids which are capable of specifically hybridizing to a nucleic acid sequence which is adjacent to a polymorphic region of interest or to a polymorphic region and are extended. A primer can be used alone in a detection method, or a primer can be used together with at least one other primer or probe in a detection method. Primers can also be used to amplify at least a portion of a nucleic acid. For amplifying at least a portion of a nucleic acid, a forward primer (i.e., 5′ primer) and a reverse primer (i.e., 3′ primer) will preferably be used. Forward and reverse primers hybridize to complementary stands of a double stranded nucleic acid, such that upon extension from each primer, a double stranded nucleic acid is amplified.

Probes refer to nucleic acids which hybridize to the region of interest and which are not further extended. For example, a probe is a nucleic acid which hybridizes adjacent to or at a polymorphic region of a COX6B gene, a GPI-1 gene or another gene associated with cardiovascular disease and which by hybridization or absence of hybridization to the DNA of a subject will be indicative of the identity of the allelic variant of the polymorphic region of the gene. Preferred probes have a number of nucleotides sufficient to allow specific hybridization to the target nucleotide sequence. Where the target nucleotide sequence is present in a large fragment of DNA, such as a genomic DNA fragment of several tens or hundreds of kilobases, the size of a probe may have to be longer to provide sufficiently specific hybridization, as compared to a probe which is used to detect a target sequence which is present in a shorter fragment of DNA. For example, in some diagnostic methods, a portion of a COX6B gene, a GPI-1 gene or another gene associated with cardiovascular disease may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

Preferred primers and probes hybridize adjacent to or at the polymorphic sites described in TABLES 1-3. In addition, preferred primers include SEQ ID NOS.: 5, 10, 43, 48, 53, 58, 63, 68, 73, 78, 83, 88, 93, 98, 103, 108, 113, and 118.

Primers and probes (RNA, DNA (single-stranded or double-stranded), PNA and their analogs) described herein may be labeled with any detectable reporter or signal moiety including, but not limited to radioisotopes, enzymes, antigens, antibodies, spectrophotometric reagents, chemiluminescent reagents, fluorescent and any other light producing chemicals. Additionally, these probes may be modified without changing the substance of their purpose by terminal addition of nucleotides designed to incorporate restriction sites or other useful sequences, proteins, signal generating ligands such as acridinium esters, and/or paramagnetic particles.

These probes may also be modified by the addition of a capture moiety (including, but not limited to para-magnetic particles, biotin, fluorescein, dioxigenin, antigens, antibodies) or attached to the walls of microtiter trays to assist in the solid phase capture and purification of these probes and any DNA or RNA hybridized to these probes. Fluorescein may be used as a signal moiety as well as a capture moiety, the latter by interacting with an anti-fluorescein antibody.

Any probe or primer can be prepared according to methods well known in the art and described, e.g., in Sambrook, J. Fritsch, E. F., and Maniatis, T. (1989( Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, probes and primers can be prepared using the Polymerase Chain Reaction (PCR) using primers having an appropriate sequence.

Oligonucleotides may be synthesized by standard methods known in the art, e.g. by use of an automated DNA synthesizer (such as are commercially available from Biosearch (Novato, Calif.); Applied Biosystems (Foster City, Calif.), etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

H. Transgenic Animals

Methods for making transgenic animals using a variety of transgenes have been described in Wagner et al. (1981) Proc. Nat. Acad. Sc. U.S.A. 78: 5016; Stewart et al. (1982) Science 217: 1046; Constantini et al. (1981) Nature 294: 92; Lacy et al. (1983) Cell 34: 343; McKnight et al. (1983) Cell 34: 335; Brinstar et al. (1983) Nature 306: 332; Palmiter et al. (1982) Nature 300: 611; Palmiter et al. (1982) Cell 29: 701; and Palmiter et al. (1983) Science 222: 809. Such methods are described in U.S. Pat. Nos. 6,175,057; 6,180,849 and 6,133,502.

The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, preferably a permanent genetic change, is induced in a cell following incorporation of exogenous DNA. A permanent genetic change is generally achieved by introduction of the DNA into the genome of the cell. Vectors for stable integration include, but are not limited to, plasmids, retroviruses and other animal viruses and YACS. Of interest are transgenic mammals, including, but are not limited to, cows, pigs, goats, horses and others, and particularly rodents, including rats and mice. Preferably, the transgenic-animals are mice.

Transgenic animals contain an exogenous nucleic acid sequence present as an extrachromosomal element or stably integrated in all or a portion of its cells, especially germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

The exogenous gene is usually either from a different species than the animal host, or is otherwise altered in its coding or non-coding sequence. The introduced gene may be a wild-type gene, naturally occurring polymorphism (e.g., as described for COX6B, GPI-1 and other genes associated with cardiovascular disease) or a genetically manipulated sequence, for example having deletions, substitutions or insertions in the coding or non-coding regions. When the introduced gene is a coding sequence, it is usually operably linked to a promoter, which may be constitutive or inducible, and other regulatory sequences required for expression in the host animal.

Transgenic animals can comprise other genetic alterations in addition to the presence of alleles of COX6B and/or GPI-1 genes. For example, the genome can be altered to affect the function of the endogenous genes, contain marker genes, or contain other genetic alterations (e.g., alleles of other genes associated with cardiovascular disease).

A “knock-out” of a gene means an alteration in the sequence of the gene that results in a decrease of function of the target gene, preferably such that target gene expression is undetectable or insignificant. A knock-out of an endogenous COX6B or GPI-1 gene means that function of the gene has been substantially decreased so that expression is not detectable or only present at insignificant levels. “Knock-out” transgenics can be transgenic animals having a heterozygous knock-out of the COX6B or GPI-1 gene or a homozygous knock-out of one or both of these genes. “Knock-outs” also include conditional knock-outs, where alteration of the target gene can occur upon, for example, exposure of the animal to a substance that promotes target gene alteration, introduction of an enzyme hat promotes recombination at the target gene site (e.g., Cre in the Cre-lox system), or other method for directing the target gene alteration postnatally.

A “knock-in” of a target gene means an alteration in a host cell genome that results in altered expression (e.g., increased (including ectopic)) of the target gene, e.g., by introduction of an additional copy of the target gene, or by operatively inserting a regulatory sequence that provides for enhanced expression of an endogenous copy of the target gene. “Knock-in” transgenics of interest can be transgenic animals having a knock-in of the COX6B or GPI-1. Such transgenics can be heterozygous or homozygous for the knock-in gene. “Knock-ins” also encompass conditional knock-ins.

A construct is suitable for use in the generation of transgenic animals if it allows the desired level of expression of a COX6B or GPI-1 encoding sequence or the encoding sequence of another gene associated with cardiovascular disease. Methods of isolating and cloning a desired sequence, as well as suitable constructs for expression of a selected sequence in a host animal, are well known in the art and are described below.

For the introduction of a gene into the subject animal, it is generally advantageous to use the gene as a gene construct wherein the gene is ligated downstream of a promoter capable of and operably linked to expressing the gene in the subject animal cells. Specifically, a transgenic non-human mammal showing high expression of the desired gene can be created by microinjecting a vector ligated with said gene into a fertilized egg of the subject non-human mammal (e.g., rat fertilized egg) downstream of various promoters capable of expressing the protein and/or the corresponding protein derived from various mammals (rabbits, dogs, cats, guinea pigs, hamsters, rats, mice etc., preferably rats etc.) Useful vectors include Escherichia coli-derived plasmids, Bacillus subtilis-derived plasmids, yeast-derived plasmids, bacteriophages such as lambda, phage, retroviruses such as Moloney leukemia virus, and animal viruses such as vaccinia virus or baculovirus.

Useful promoters for such gene expression regulation include, for example, promoters for genes derived from viruses (cytomegalovirus, Moloney leukemia virus, JC virus, breast cancer virus etc.), and promoters for genes derived from various mammals (humans, rabbits, dogs, cats, guinea pigs, hamsters, rats, mice etc.) and birds (chickens etc.) (e.g., genes for albumin, insulin II, erythropoietin, endothelin, osteocalcin, muscular creatine kinase, platelet-derived growth factor beta, keratins K1, K10 and K14, collagen types I and II, atrial natriuretic factor, dopamine beta-hydroxylase, endothelial receptor tyrosine kinase (generally abbreviated Tie2), sodium-potassium adenosine triphosphorylase (generally abbreviated Na,K-ATPase), neurofilament light chain, metallothioneins I and IIA, metalloproteinase I tissue inhibitor, MHC class I antigen (generally abbreviated H-2L), smooth muscle alpha actin, polypeptide chain elongation factor 1 alpha (EF-1 alpha), beta actin, alpha and beta myosin heavy chains, myosin light chains 1 and 2, myelin base protein, serum amyloid component, myoglobin, renin etc.).

It is preferable that the above-mentioned vectors have a sequence for terminating the transcription of the desired messenger RNA in the transgenic animal (generally referred to as terminator); for example, gene expression can be manipulated using a sequence with such function contained in various genes derived from viruses, mammals and birds. Preferably, the simian virus SV40 terminator etc. are commonly used. Additionally, for the purpose of increasing the expression of the desired gene, the splicing signal and enhancer region of each gene, a portion of the intron of a eukaryotic organism gene may be ligated 5′ upstream of the promoter region, or between the promoter region and the translational region, or 3′ downstream of the translational region as desired.

A translational region for a protein of interest can be obtained using the entire or portion of genomic DNA of blood, kidney or fibroblast origin from various mammals (humans, rabbits, dogs, cats, guinea pigs, hamsters, rats, mice etc.) or of various commercially available genomic DNA libraries, as a starting material, or using complementary DNA prepared by a known method from RNA of blood, kidney or fibroblast origin as a starting material. Also, an exogenous gene can be obtained using complementary DNA prepared by a known method from RNA of human fibroblast origin as a starting material. All these translational regions can be utilized in transgenic animals.

To obtain the translational region, it is possible to prepare DNA incorporating an exogenous gene encoding the protein of interest in which the gene is ligated downstream of the above-mentioned promoter (preferably upstream of the translation termination site) as a gene construct capable of being expressed in the transgenic animal.

DNA constructs for random integration need not include regions of homology to mediate recombination. Where homologous recombination is desired, the DNA constructs will comprise at least a portion of the target gene with the desired genetic modification, and will include regions of homology to the target locus. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

The transgenic animal can be created by introducing a COX6B or GPI-1 gene construct into, for example, an unfertilized egg, a fertilized egg, a spermatozoon or a germinal cell containing a primordial germinal cell thereof, preferably in the embryogenic stage in the development of a non-human mammal (more preferably in the single-cell or fertilized cell stage and generally before the 8-cell phase), by standard means, such as the calcium phosphate method, the electric pulse method, the lipofection method, the agglutination method, the microinjection method, the particle gun method, the DEAE-dextran method and other such method. Also, it is possible to introduce a desired COX6B or GPI-1 gene into a somatic cell, a living organ, a tissue cell, or the like, by gene transformation methods, and utilize it for cell culture, tissue culture etc. Furthermore, these cells may be fused with the above-described germinal cell by a commonly known cell fusion method to create a transgenic animal.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected. The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture.

Animals containing more than one transgene, such as allelic variants of COX6B and/or GPI-1 and/or other genes associated with cardiovascular disease can be made by sequentially introducing individual alleles into an animal in order to produce the desired phenotype (manifestation or predisposition to cardiovascular disease).

I. Effect of Allelic Variants on the Encoded Protein and Disease Related Phenotype

The effect of an allelic variant on a COX6B or GPI-1 protein (altered amount, stability, location and/or activity) can be determined according to methods known in the art. Alielic variants of the COX6B and GPI-1 genes can be assayed individually or in combination with other variants known to be associated with cardiovascular disease.

If the mutation is located in an intron, the effect of the mutation can be determined, e.g., by producing transgenic animals in which the allelic variant linked to lipid metabolism and/or cardiovascular disease has been introduced and in which the wild-type gene or predominant allele may have been knocked out. Comparison of the level of expression of the protein in the mice transgenic for the allelic variant with mice transgenic for the predominant allele will reveal whether the mutation results in increased or decreased synthesis of the associated protein and/or aberrant tissue distribution of the associated protein. Such analysis could also be performed in cultured cells, in which the human variant allele gene is introduced and, e.g., replaces the endogenous gene in the cell. Thus, depending on the effect of the alteration a specific treatment can be administered to a subject having such a mutation. Accordingly, if the mutation results in decreased production of a COX6B or GPI-1 protein, the subject can be treated by administration of a compound which increases synthesis, such as by increasing COX6B or GPI-1 gene expression, and wherein the compound acts at a regulatory element different from the one which is mutated. Alternatively, if the mutation results in increased COX6B or GPI-1 protein levels, the subject can be treated by administration of a compound which reduces protein production, e.g., by reducing COX6B or GPI-1 gene expression or a compound which inhibits or reduces the activity of COX6B or GPI-1 protein.

J. Diagnostic and Prognostic Assays

Typically, an individual allelic variant that associates with a risk factor for cardiovascular disease will not be used in isolation as a prognosticator for a subject developing high cholesterol, low HDL or cardiovascular disease. An allelic variant typically will be one of a plurality of indicators that are utilized. The other indicators may be the manifestation of other risk factors for cardiovascular disease, e.g., family history, high blood pressure, weight, activity level, etc., or additional allelic variants in the same or other genes associated with altered lipid metabolism and/or cardiovascular disease.

Useful combinations of allelic variants of the COX6B gene and/or the GPI-1 gene can be determined by examining combinations of variants of these genes, which are assayed individually or assayed simultaneously using multiplexing methods as described above or any other labelling method that allows different variants to be identified. In particular, variants of COX6B gene and/or the GPI-1 gene may be assayed using kits (see below) or any of a variety microarrays known to those in the art. For example, oligonucleotide probes comprising the polymorphic regions surrounding any polymorphism in the COX6B or GPI-1 gene may be designed and fabricated using methods such as those described in U.S. Pat. Nos. 5,492,806; 5,525,464; 5,695,940; 6,018,041; 6,025,136; WO 98/30883; WO 98/56954; WO99/09218; WO 00/58516; WO 00/58519, or references cited therein. Similarly one of skill in the art can determine useful combinations of allelic variants of the COX6B and/or GPI-1 genes along with variants of other genes associated with cardiovascular disease.

K. Pharmacogenomics

It is likely that subjects having one or more different allelic variants of the COX6B or GPI-1 polymorphic regions will respond differently to therapeutic drugs to treat cardiovascular disease or conditions. For example, there are numerous drugs available for lowering cholesterol levels: including lovastatin (MEVACOR; Merck & Co.), simvastatin (XOCOR; Merck & Co.), dextrothyroxine (CHOLOXIN; Knoll Pharmaceutical Co.), pamaqueside (Pfizer), cholestryramine (QUESTRAN; Bristol-Myers Squibb), colestipol (COLESTID; Pharmacia & Upjohn), acipomox (Pharmacia & Upjohn), fenofibrate (LIPIDIL), gemfibrozil (LOPID; Warner-Lambert), cerivastatin (LIPOBAY; Bayer), fluvastatin (LESCOL; Novartis), atorvastatin (LIPITOR, Warner-Lambert), etofylline clofibrate (DUOLIP; Merckle (Germany)), probucol (LORELCO; Hoechst Marion Roussel), omacor (Pronova (Norway), etofibrate (Merz (Germany), clofibrate (ATROMID-S; Wyeth-Ayerst (AHP)), and niacin (numerous manufacturers). All patients do not respond identically to these drugs. Alleles of the COX6B or the GPI-1 gene which associate with altered lipid metabolism will be useful alone or in conjunction with markers in other genes associated with the development of cardiovascular disease to predict a subject's response to a therapeutic drug. For example, multiplex primer extension assays or microarrays comprising probes for alleles are useful formats for determining drug response. A correlation between drug responses and specific alleles or combinations of alleles of the COX6B or GPI-1 genes and other genes associated with cardiovascular disease can be shown, for example, by clinical studies wherein the response to specific drugs of subjects having different allelic variants of polymorphic regions of the COX6B or GPI-1 genes alone or in combination with allelic variants of other genes are compared. Such studies can also be performed using animal models, such as mice having various alleles and in which, e.g., the endogenous COX6B or GPI-1 genes have been inactivated such as by a knock-out mutation. Test drugs are then administered to the mice having different alleles and the response of the different mice to a specific compound is compared. Accordingly, assays, microarrays and kits are provided for determining the drug which will be best suited for treating a specific disease or condition in a subject based on the individual's genotype. For example, it will be possible to select drugs which will be devoid of toxicity, or have the lowest level of toxicity possible for treating a subject having a disease or condition, e.g., cardiovascular disease or high cholesterol or low HDL.

L. Kits

Kits can be used to indicate whether a subject is at risk of developing high cholesterol, low HDL and/or cardiovascular disease. The kits can also be used to determine if a subject who has high cholesterol or low HDL carries associated variants in the COX6B or GPI-1 genes or other cardiovascular disease-related genes. This information could be used, e.g., to optimize treatment of such individuals as a particular genotype may be associated with drug response.

In preferred embodiments, the kits comprise a probe or primer which is capable of hybridizing adjacent to or at a polymorphic region of a OX6B or GPI-1 gene and thereby identifying whether the COX6B or GPI-1 gene contains an allelic variant which is associated with cardiovascular disease. Primers or probes that specifically hybridize at or adjacent to the SNPs described in Tables 1-3 could be included. In particular, primers or probes which comprise the sequences of SEQ ID NOs.: 5, 10, 43, 48, 53, 58, 63, 68, 73, 78, 83, 88, 93, 98, 103, 108, 113, and 118 could be included in the kits. The kits preferably further comprise instructions for use in carrying out assays, interpreting results and diagnosing a subject as having a predisposition toward developing high cholesterol, low HDL and/or cardiovascular disease.

Preferred kits for amplifying a region of a COX6B gene, GPI-1 gene, or other genes associated with cardiovascular disease (such as those listed in Table 3) comprise two primers which flank a polymorphic region of the gene of interest. For example primers can comprise the sequences of SEQ ID NOs.: 3, 4, 8, 9, 41, 42, 46, 47, 51, 52, 56, 57, 61, 62, 66, 67, 71, 72, 76, 77, 81, 82, 86, 87, 91, 92, 96, 97, 101, 102, 106, 107, 111, 112, 116, and 117. For other assays, primers or probes hybridize to a polymorphic region or 5′ or 3′ to a polymorphic region depending on which strand of the target nucleic acid is used. For example, specific probes and primers comprise sequences designated as SEQ ID NOs: 5, 10, 43, 48, 53, 58, 63, 68, 73, 78, 83, 88, 93, 98, 103, 108, 113, and 118. Those of skill in the art can synthesize primers and probes which hybridize adjacent to or at the polymorphic regions described in TABLES 1-3 and other SNPs in genes associated with cardiovascular disease.

Yet other kits comprise at least one reagent necessary to perform an assay. For example, the kit can comprise an enzyme, such as a nucleic acid polymerase. Alternatively the kit can comprise a buffer or any other necessary reagent.

Yet other kits comprise microarrays of probes to detect allelic variants of COX6B, GPI-1, and other genes associated with cardiovascular disease. The kits further comprise instructions for their use and interpreting the results.

The following examples are included for illustrative purposes only and are not intended to limit the scope of the invention. The practice of methods and development of the products provided herein employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide To Molecular Cloning (1984); the treatise, Methods In Enzymology (Academic Press, Inc., New York); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds., Immunochemical Methods In Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLE 1

Isolation of DNA from Blood Samples of a Stratified Population

Blood samples were obtained from a population of unrelated Caucasian women between the ages of 18-79 (average age=48). The women had, no response to media campaigns, attended the Twin Research Unit at the St. Thomas Hospital in London, England. For current purposes, only one member of a twin pair was used to insure that all observations were independent. Blood samples from 1400 unrelated individuals were measured for levels of cholesterol and HDL. Cholesterol and HDL level in blood samples were quantitated using standard assay methods.

The population was stratified into pools of 200 people, which represented the lower extreme and the upper extreme for serum levels of cholesterol and HDL.

Cholesterol

| | |
|---|---|
| Pool 1: | Individuals were considered to have low |
| | cholesterol (0.12-3.6 mmoles/L). |
| Pool 2: | Individuals were considered to have high |
| | cholesterol (5.25-11.57 mmoles/L). |

HDL

| | |
|---|---|
| Pool 3: | Individuals were considered to have low levels |
| | of HDL (0.240-1.11 mmoles/L) |
| Pool 4: | Individuals were considered to have high levels |
| | of HDL (2.10-3.76 mmoles/L). |

DNA Extraction Protocol

DNA was extracted from blood samples of each of the pools by utilizing the following protocol.

Section 1

1. Blood was extracted into EDTA tubes.

2. Blood sample was spun at 3,000 rpm for 10 minutes in a clinical centrifuge.

3. The buffy coat (the leukocytes, a yellowish layer of cells on top of the red blood cells) was removed and pooled into a 1 ml conical tube.

4. 0.9% saline was added to fill the tube and resuspend the leucocytes. Sample were immediately further processed or stored at 4° C. for 24 hrs.

5. The sample was spun at 2,500 rpm for 10 minutes.

6. The buffy coat was again removed as cleanly as possible leaving behind any red cells, the sample was suspended in red cell lysis buffer and left for 20 minutes at 4° C.

7. The sample was spun again at 2,500 rpm for 10 minutes. If a pellet of unlysed red cells remained lying above the leucocytes the treatment with red cell lysis buffer was repeated.

8. The leucocyte pellet was resuspended in 2 ml 0.9% saline.

9. The DNA was liberated by the addition of leucocyte lysis buffer—the tube was capped and gently inverted several times, until the liquid became viscous with DNA. The samples were handled with care to avoid shearing and damage to the DNA.

10. Samples were frozen for storage prior to full extraction.

Section 2

11. 2 ml of 5 M sodium perchlorate was added to the thawed sample and mixed by inversion. The sample was heated to 60° C. for 30-40 minutes to fully denature proteins.

12. An equal volume of chloroform/isoamyl alcohol (24:1) was added at room temperature and the sample mixed for 10 minutes.

13. The sample was spun without a break at 3,000 rpm for 10 minutes.

14. The top aqueous phase was removed into a clean tube and two volumes of cold 100% ethanol added and mixed by inversion to precipitate DNA.

15. The DNA was removed using a sterile loop and resuspended in 1-5 ml TE buffer depending on the DNA yield.

16. The optical density was measured at 260 and 280 nm to check yield and purity of the DNA sample. For use in Examples 2 and 3, all DNA had an absorbance ratio of 1.6 at 260/280, a total yield of 32 μg and a concentration of 10 ng/μl. If initial purity levels were unacceptable a re-extraction was carried out (sections 12-15 above).

### EXAMPLE 2

Detection of an Association Between an SNP at Position 86 of the Human COX6B Gene and High Cholesterol

DNA samples (as prepared in Example 1), representing 200 women, from the lower extreme, pool 1 (low levels of cholesterol) and the upper extreme, pool 2 (high levels of cholesterol) were amplified and analyzed for genetic differences using a MassEXTEND™ assay detection method. For each pool, single nucleotide polymorphisms were examined throughout the entire genome to detect differences in allelic frequency of a variant allele between the pools.

PCR Amplification of Samples from Pools 1 and 2

PCR primers were synthesized by Operon (Alameda, Calif.) using phosphoramidite chemistry. Amplification of the COX6B target sequence was carried out in two 50 μl PCR reactions with 100 ng of pooled human genomic DNA, obtained as described in Example 1, taken from samples in pool 1 or pool 2, although amounts ranging from 100 ng to 1 ug could be used. Individual DNA concentrations within the pooled samples were present in equal concentration with a final concentration of 0.5 ng. Each reaction contained 1×PCR buffer (Qiagen, Valencia, Calif.), 200 μM dNTPs, 1U Hotstar Taq polymerase (Qiagen, Valencia, Calif.), 4 mM MgCl₂, and 25 pmols of the long primer containing both the universal primer sequence and the target specific sequence 5′-AGCGGATAACAATTTCACACAGGTAGTCTGGTTCTGGTTGGGG-3′ (SEQ ID NO.: 4), 2 pmoles of the short primer 5′-AGGATTCAGCACCATGGC-3′ (SEQ ID NO.: 3) and 10 pmoles of a biotinylated universal primer complementary to the 5′ end of the PCR amplicon 5′-AGCGGATAACAATTTCACACAGG-3′ (SEQ ID NO.: 121). Alternatively, the biotinylated universal primer could be 5′-GGCGCACGCCTCCACG-3′ (SEQ ID NO.: 122). After an initial round of amplification with the target with the specific forward (long) and reverse primer (short), the 5′ biotinylated universal primer then hybridized and acted as a reverse primer thereby introducing a 3′ biotin capture moiety into the molecule. The amplification protocol results in a 5′-biotinylated double stranded DNA amplicon and dramatically reduces the cost of high throughput genotyping by eliminating the need to 5′ biotin label each forward primer used in a genotyping. Thermal cycling was performed in 0.2 mL tubes or 96 well plate using an MJ Research Thermal Cycler (Waltham, Mass.) (calculated temperature) with the following cycling parameters: 94° C. for 5 min; 45 cycles: 94° C. for 20 sec, 56° C. for 30 sec, 72° C. for 60 sec; 72° C. 3 min.

Immobilization of DNA

The 50 μl PCR reaction was added to 25 μl of streptavidin coated magnetic bead (Dynal, Lake Success, N.Y.) prewashed three times and resuspended in 1 M NH₄Cl, 0.06 M NH₄OH. The PCR amplicons were allowed to bind to the beads for 15 minutes at room temperature. The beads were then collected with a magnet and the supernatant containing unbound DNA was removed. The unbound strand was released from the double stranded amplicons by incubation in 100 mM NaOH and washing of the beads three times with 10 mM Tris pH 8.0.

Genotyping

The frequency of the alleles at position 86 in the COX6B gene was measured using the MassEXTEND™ assay and MALDI-TOF. The SNP identified at position 86 of COX6B in the GenBank sequence is represented as a C to T transversion. The MassEXTEND™ assay used detected the sequence of the complementary strand, thus the SNP was represented as G to A in the primer extension products. The DNA coated magnetic beads were resuspended in 26 mM Tris-HCL pH 9.5, 6.5 mM MgCl₂ and 50 mM each of dTTPs and 50 mM each of ddCTP, ddATP, ddGTP, 2.5 U of a thermostable DNA polymerase (Amersham Pharmacia Biotech, Piscataway, N.J.) and 20 pmoles of a template specific oligonucleotide primer 5′-AATCAAGAACTACAAGAC-3′ (SEQ ID NO.: 5) (Operon, Alameda, Calif.). Primer extension occurred with three cycles of oligonucleotide primer hybridization and extension. The extension products were analyzed after denaturation from the template with 50 mM NH₄Cl and transfer of 150 nl of each sample to a silicon chip preloaded with 150 nl of H3PA (3-hydroxy picolinic acid) (Sigma Aldrich, St Louis, Mo.) matrix material. The sample material was allowed to crystallize and analyzed by MALDI-TOF (Bruker Daltonics, Billerica, Mass.; PerSeptive, Foster City, Calif.). The mass of the primer used in the MassEXTEND™ reaction was 5493.70 daltons. The predominant allele is extended by the addition of ddC, which has a mass of 5766.90 daltons. The allelic variant results in the addition of dT and ddG to the primer to produce an extension product having a mass of 6111.10 daltons.

In addition to being analyzed as part of a pool, each individual sample (0.5 ng) was amplified as described above and analyzed individually using a MassEXTEND™ reaction as described above.

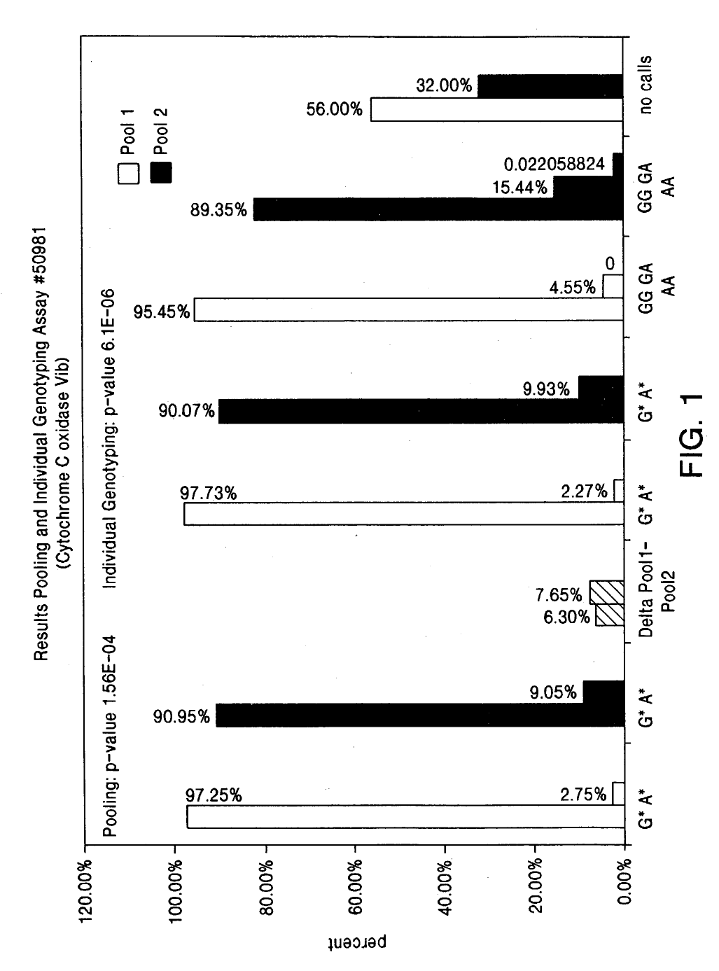

Pooled populations of women (200 women per pool) with high cholesterol (pool 2) showed an increase in the frequency of the A allele at nucleotide position 86 of COX6B as compared with those with low levels of cholesterol (pool 1) (see FIG. 1). The association of this allelic variant of the COX6B gene with high cholesterol gave a statistically significant value of 14.30 using a 1-degree-of-freedom chi-squared test of association. In other words, the increase of 2.75% to 9.05% is significant, with a p value of 0.000156 (see FIG. 1). The genotype of each of the individuals in the pooled population was also determined by carrying out MassEXTEND™ reactions on each DNA samples individually. These analysis confirmed the pooling data showing that there was an increase in the frequency of the A allele of 2.27% to 9.93%, (p=0.0000061). The genotypes in pool 2 showed a decrease in the homozygous GG genotype from 95.4% to 82.35% and an increase in the heterozygous GA genotype from 4.55% to 15.44%. None of the individuals with low levels of serum cholesterol exhibited the homozygous AA genotype.

### EXAMPLE 3

Detection of an Association Between an SNP at Position 2577 of the Human GPI-1 Gene and Low HDL

DNA samples (as prepared in Example 1), representing 200 women, from pool 3 (low level of HDL) and pool 4 (high levels of HDL) were amplified and analyzed for genetic differences using a MassEXTEND™ detection method. For each pool, SNPs were examined throughout the genome to detect differences in allelic frequency of variant alleles between the pools.

PCR Amplification of Samples from Pools 3 and 4

PCR primers were synthesized by Operon (Alameda, Calif.) using phosphoramidite chemistry. Amplification of the GPI-1 target sequence was carried out in single 50 μl PCR reaction with 100 ng of pooled human genomic DNA (200 samples), obtained as described in Example 1, taken from samples in pool 3 or pool 4, although amounts ranging from 100 ng to 1 ug could be used. Individual DNA concentrations within the pooled samples were present in equal concentration with the final concentration of 0.5 ng. Each reaction contained 1×PCR buffer (Qiagen, Valencia, Calif.), 200 uM dNTPs, 1U Hotstar Taq polymerase (Qiagen, Valencia, Calif.), 4 mM MgCl₂, and 25 pmols of the forward primer containing both the universal primer sequence and the target specific short sequence 5′-AGCAGGGCTTCCTCCTTC-3′ (SEQ ID NO.: 8) 2 pmoles of the long primer 5′-AGCGGATAACAATTTCACACAGGTGACCCAGCCGTACCTATTC-3′ (SEQ ID NO.: 9) and 10 pmoles of a biotinylated universal primer complementary to the 5′ end of the PCR amplicon 5′-AGCGGATAACAATTTCACACAGG-3′ (SEQ ID NO.: 121). After an initial round of amplification with the target with the specific forward (long) and reverse primer (short), the 5′ biotinylated universal primer then hybridized and acted as a reverse primer thereby introducing a 3′ biotin capture moiety into the molecule. The amplification protocol results in a 5′-biotinylated double stranded DNA amplicon and dramatically reduces the cost of high throughput genotyping by eliminating the need to 5′ biotin label each forward primer used in a genotyping. Thermal cycling was performed in 0.2 mL tubes or 96 well plate using an MJ Research Thermal Cycler (Watham, Mass.) (calculated temperature) with the following cycling parameters: 94° C. for 5 min; 45 cycles: 94° C. for 20 sec, 56° C. for 30 sec, 72° C. for 60 sec; 72° C. 3 min.

Immobilization of DNA

The 50 μl PCR reaction was added to 25 μl of streptavidin coated magnetic bead (Dynal, Lake Success, N.Y.) prewashed three times and resuspended in 1 M NH₄Cl, 0.06 M NH₄OH. The PCR amplicons were allowed to bind to the beads for 15 minutes at room temperature. The beads were then collected with a magnet and the supernatant containing unbound DNA was removed. The unbound strand was released from the double stranded amplicons by incubation in 100 mM NaOH and washing of the beads three times with 10 mM Tris pH 8.0.

Genotyping

The frequency of the alleles at position 2577 in the GPI-1 gene was measured using the MassEXTEND™ assay and MALDI-TOF. The SNP identified at position 2577 of GPI-1 in the GenBank sequence is represented as a G to A transversion. The MassEXTEND™ assay used detected this sequence, thus the SNP was represented as C to T in the primer extension products. The DNA coated magnetic beads were resuspended in 26 mM Tris-HCL pH 9.5, 6.5 mM MgCl₂ and 50 mM each of dTTPs and 50 mM each of ddCTP, ddATP, ddGTP, 2.5 U of a thermostable DNA polymerase (Amersham Pharmacia Biotech, Piscataway, N.J.) and 20 pmoles of a template specific oligonucleotide primer 5′-AAGGGAGACAGATTTGGC-3′ (SEQ ID NO.: 10) (Operon, Alameda, Calif.). Primer extension occurred with three cycles of oligonucleotide primer hybridization and extension. The extension products were analyzed after denaturation from the template with 50 mM NH₄Cl and transfer of 150 nl each sample to a silicon chip preloaded with 150 nl of H3PA matrix material. The sample material was allowed to crystallize and analyzed by MALDI-TOF (Bruker Daltonics, Billerica, Mass.; PerSeptive, Foster City, Calif.). The mass of the primer used in the MassEXTEND™ reaction was 561 2.70 daltons. The predominant allele is extended by the addition of ddC, which has a mass of 5885.90 daltons. The allelic variant results in the addition of dT and ddG to the primer to produce an extension product having a mass of 6230.10 daltons.

In addition to being analyzed as a pool, each individual sample (0.5 ng) was amplified as described above and analyzed individually using the MassEXTEND™ reaction as described above.

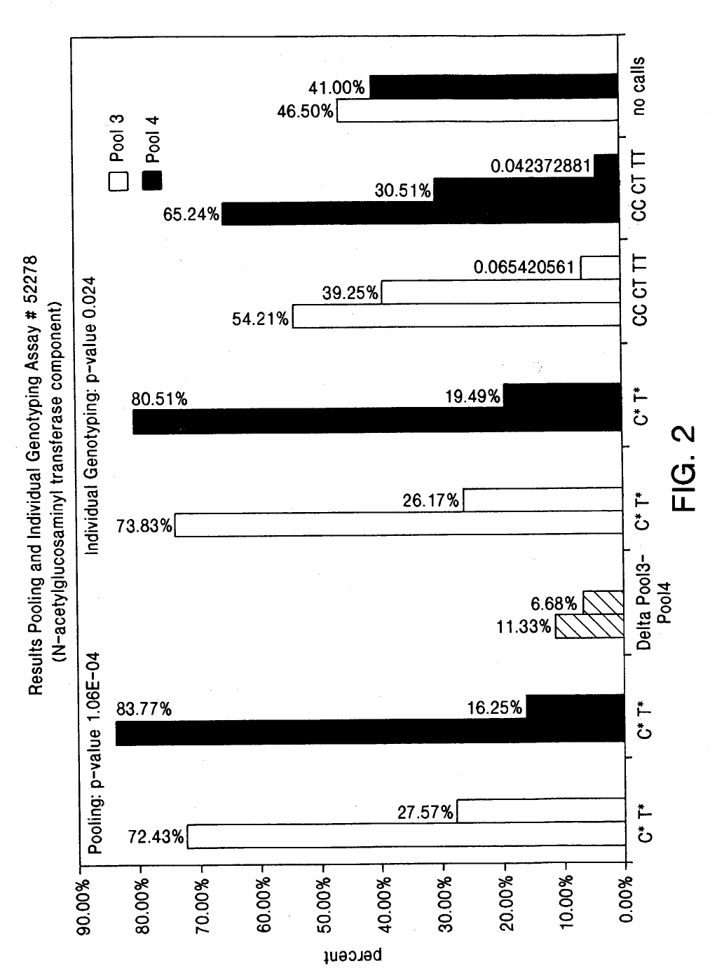

Pooled populations of women (200 women per pool) with low HDL (pool 3) showed an increase in the T allele of 11.33% at nucleotide position 2577 as compared with those with high levels of HDL (pool 4). The association of this allelic variant of the GPI-1 gene with low HDL gave a statistically significant value of 15.04 using a 1-degree-of-freedom chi-squared test of association. In other words, the increase of 16.23% to 27.57% is significant, with a p value of 0.0001064 (see FIG. 2). The genotype of each of the individuals in the pooled population was also determined by carrying out individual MassEXTEND™ reactions on individual DNA samples. These analysis confirmed the pooling data showing that there was an increase in the frequency of the T allele of 19.49% to 26.1%, (p=0.024). The measured genotypes in pool 3 showed a decrease in the homozygous CC genotype from 65.24% to 54.21% and an increase in the heterozygous CT genotype from 30.51% to 39.25%. The homozygous TT genotypes increased 2.3%.

Since modifications will be apparent to those of skill in this art, it is intended that this invention be limited only by the scope of the appended claims.

What is claimed:

1. A method for detecting the presence or absence in a subject of at least one allelic variant of a polymorphic region of a gene associated with cardiovascular disease, comprising:
the step of detecting the presence or absence of an allelic variant of a polymorphic region of a cytochrome C oxidase subunit VIb (COX6B) gene of the subject that is associated with high serum cholesterol or an allelic variant of a polymorphic region of a N-acetylglucosaminyl transferase component GPI-1 (GPI-1) gene of the subject that is associated with low serum high density lipoprotein (HDL).

2. The method of claim 1, wherein the allelic variant is of a polymorphic region of the N-acetylglucosaminyl transferase component GPI-1 (GPI-1) gene.

3. The method of claim 1, further comprising detecting the presence or absence in a subject of least one allelic variant of another gene associated with cardiovascular disease.

4. The method of claim 3, wherein the other gene is selected from the group consisting of cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase, E-selectin, G protein beta 3 subunit and angiotensin II type 1 receptor gene.

5. The method of claim 2, wherein the polymorphic region is a single nucleotide polymorphism (SNP).

6. The method of claim 5, wherein the SNP is at position 2577 of the N-acetylglucosaminyl transferase component GPI-1 (GPI-1) gene sequence and the allelic variant is represented by an A nucleotide in the sense strand or a T nucleotide in the corresponding position in the antisense strand.

7. The method of claim 1, wherein the detecting step is by a method selected from the group consisting of allele specific hybridization, primer specific extension, oligonucleotide ligation assay, restriction enzyme site analysis and single-stranded conformation polymorphism analysis.

8. The method of claim 6, further comprising:
(a) hybridizing a target nucleic acid comprising a N-acetylglucosaminyl transferase component GPI-1 (GPI-1)-encoding nucleic acid or fragment thereof with a nucleic acid primer that hybridizes adjacent to nucleotide 2577 of the GPI-1 gene;
(b) extending the nucleic acid primer using the target nucleic acid as a template; and
(c) determining the mass of the extended primer to identify the nucleotide present at position 2577, thereby determining the presence or absence of the allelic variant.

9. The method of claim 1, wherein the detecting step comprises mass spectrometry.

10. The method of claim 1, wherein the detecting step utilizes a signal moiety selected from the group consisting of: radioisotopes, enzymes, antigens, antibodies, spectrophotometric reagents, chemiluminescent reagents, fluorescent reagents and other light producing reagents.

11. The method of claim 8, wherein the nucleic acid primer is extended in the presence of at least one dideoxynucleotide.

12. The method of claim 11, wherein the dideoxynucleotide is dideoxyguanosine (ddG).

13. The method of claim 8, wherein the primer is extended in the presence of at least two dideoxynucleotides and the dideoxynucleotides are dideoxyguanosine (ddG) and dideoxycytosine (ddC).

14. A method for indicating a predisposition to cardiovascular disease in a subject, comprising:
the step of detecting in a target nucleic acid obtained from the subject the presence or absence of at least one allelic variant of polymorphic regions of a cytochrome C oxidase subunit VIb (COX6B) gene associated with high serum cholesterol or at least one allelic variant of polymorphic regions of a N-acetylglucosaminyl transferase component GPI-1 (GPI-1) gene associated with low serum HDL wherein the presence of an allelic variant is indicative of a predisposition to cardiovascular disease compared to a subject who does not comprise the allelic variant.

15. The method of claim 14, wherein the allelic variant is of a polymorphic region of the N-acetylglucosaminyl transferase component GPI-1 (GPI-1) gene.

16. The method of claim 15, wherein the polymorphic region is a single nucleotide polymorphism (SNP).

17. The method of claim 16, wherein the SNP is at position 2577 of the N-acetylglucosaminyl transferase component GPI-1 (GPI-1) gene sequence and the allelic variant is represented by an A nucleotide in the sense strand or a T nucleotide in the corresponding position in the antisense strand.

18. The method of claim 14, wherein the detecting step is by a method selected from the group consisting of allele specific hybridization, primer specific extension, oligonucleotide ligation assay, restriction enzyme site analysis and single-stranded conformation polymorphism analysis.

19. The method of claim 17, further comprising:
(a) hybridizing a target nucleic acid comprising a N-acetylglucosaminyl transferase component GPI-1 (GPI-1 )-encoding nucleic acid or fragment thereof with a nucleic acid primer that hybridizes adjacent to nucleotide 2577 of the GPI-1 gene;
(b) extending the nucleic acid primer using the target nucleic acid as a template; and
(c) determining the mass of the extended primer to identify the nucleotide present at position 2577, thereby determining the presence or absence of the allelic variant.

20. The method of claim 14, wherein the detecting step comprises mass spectrometry.

21. The method of claim 14, wherein the detecting step utilizes a signal moiety selected from the group consisting of: radioisotopes, enzymes, antigens, antibodies, spectrophotometric reagents, chemiluminescent reagents, fluorescent reagents and other light producing reagents.

22. The method of claim 14, further comprising detecting the presence or absence of at least one allelic variant of polymorphic regions of another gene associated with cardiovascular disease, wherein the presence of the two allelic variants is associated with a predisposition to cardiovascular disease compared to a subject who does not comprise the combination of allelic variants.

23. The method of claim 22, wherein the other gene is selected from the group consisting of cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase, E-selectin, G protein beta 3 subunit and angiotensin II type 1 receptor gene.

24. The method of claim 22, wherein the two allelic variants are of the cytochrome C oxidase subunit VIb (COX6B) gene and the N-acetylglucosaminyl transferase component GPI-1 (GPI-1) gene.

25. A method of screening for biologically active agents that modulate serum high density lipoprotein (HDL), comprising:
(a) combining a candidate agent with a cell comprising a nucleotide sequence encoding an allelic variant of a N-acetylglucosaminyl transferase component GPI-1 (GPI-1) gene associated with low levels of serum HDL and operably linked to a promoter such that the nucleotide sequence is expressed as a GPI-1 protein in the cell; and
(b) determining the affect of the agent upon the expression and/or activity of the GPI-1 protein.

26. A method of screening for biologically active agents that modulate serum high density lipoprotein (HDL), comprising:
(a) combining a candidate agent with a transgenic mouse comprising a transgenic nucleotide sequence stably integrated into the genome of the mouse encoding an allelic variant of a N-acetylglucosaminyl transferase component GPI-1 (GPI-1) gene associated with low levels of serum HDL operably linked to a promoter, wherein the transgenic nucleotide sequence is expressed and the transgenic animal develops a low level of serum HDL; and
(b) determining the affect of the agent upon the serum HDL level.

27. The method of claim 25, wherein the allelic variant is at position 2577 of the N-acetylglucosaminyl transferase component GPI-1 (GPI-1) gene.

28. The method of claim 26, wherein the allelic variant is at position 2577 of the N-acetylglucosaminyl transferase component GPI-1 (GPI-1) gene.

29. A method for predicting a response of a subject to a cardiovascular drug, comprising:
detecting the presence or absence of at least one allelic variant of a cytochrome C oxidase subunit VIb (COX6B) gene of the subject associated with high serum cholesterol or at least one allelic variant of a N-acetylglucosaminyl transferase component GPI-1 (GPI-1) gene of the subject associated with low serum high density lipoprotein (HDL);
wherein the presence of at least one allelic variant is indicative of a positive response.

30. The method of claim 29, wherein the allelic variant is of the N-acetylglucosaminyl transferase component GPI-1 (GPI-1) gene.

31. A method for predicting a response of a subject to a biologically active agent that modulates serum high density lipoprotein (HDL), comprising:
detecting the presence or absence of at least one allelic variant of a N-acetylglucosaminyl transferase component GPI-1 (GPI-1) gene of the subject associated with low HDL; wherein the presence of an allelic variant is indicative of a positive response.

32. A method for predicting a response of a subject to a biologically active agent that modulates serum high density lipoprotein (HDL) levels, comprising:
(a) detecting the presence or absence of at least one allelic variant of a N-acetylglucosaminyl transferase component GPI-1 (GPI-1) gene associated with low HDL of the subject; and
(b) detecting the presence or absence of an allelic variant in at least one other gene of subject associated with cardiovascular disease, wherein the presence of both allelic variants is indicative of a positive response.

33. The method of claim 31, wherein the allelic variant of a N-acetylglucosaminyl transferase component GPI-1 (GPI-1) gene is at position 2577.

34. The method of claims 32, wherein the allelic variant of a N-acetylglucosaminyl transferase component GPI-1 (GPI-1) gene is at position 2577.

35. The method of claim 32, wherein the other gene associated with cardiovascular disease is selected from the group of genes consisting of cytochrome C oxidase subunit VIb (COX6B); cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase, E-selectin, G protein beta 3 subunit and angiotensin II type I receptor gene.

36. A primer or probe that specifically hybridizes adjacent to or at a polymorphic region of a cytochrome C oxidase subunit VIb (COX6B) gene associated with high serum cholesterol in combination with a primer or probe that specifically hybridizes adjacent to or at a polymorphic region of a N-acetylglucosaminyl transferase component GPI-1 (GPI-1) gene associated with low HDL.

37. The primers or probes of claim 36, further comprising primers or probes that specifically hybridizes adjacent to or at a polymorphic region of another gene associated with cardiovascular disease.

38. The primers or probes of claim 36, wherein the polymorphic region of the cytochrome C oxidase subunit VIb (COX6B) gene comprises nucleotide 86 of the coding strand and the polymorphic region of the N-acetylglucosaminyl transferase component GPI-1 (GPI-1) gene comprises nucleotide 2577.

39. The primers or probes of claim 37, wherein the other gene associated with cardiovascular disease is selected from the group of genes consisting of cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase, E-selectin, G protein beta 3 subunit and angiotensin II type 1 receptor gene.

40. A kit for indicating whether a subject has a predisposition to developing cardiovascular disease, comprising:
(a) at least one probe or primer that specifically hybridizes adjacent to or at a polymorphic region of a N-acetylglucosaminyl transferase component GPI-1 (GPI-1) gene associated with low serum high density lipoprotein (HDL).

41. The kit of claim 40 further comprising instructions for use.

42. The kit of claim 40, wherein the polymorphic region comprises nucleotide 2577 of the coding strand.

43. A kit for indicating whether a subject has a predisposition to developing cardiovascular disease, comprising:
(a) at least one probe or primer which specifically hybridizes adjacent to or at a polymorphic region of a N-acetylglucosaminyl transferase component GPI-1 (GPI-1) gene associated with low serum high density lipoprotein (HDL); and
(b) at least one probe or primer which specifically hybridizes adjacent to or at a polymorphic region of another gene associated with cardiovascular disease.

44. The kit of claim 43, further comprising instructions for use.

45. The kit of claim 43, wherein the other gene associated with cardiovascular disease is selected from the group of genes consisting of cytochrome C oxidase subunit VIb (COX6B); cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase, E-selectin, G protein beta 3 subunit and angiotensin II type 1 receptor gene.

46. A method of diagnosing a predisposition to cardiovascular disease in a human, said method comprising the steps of:
(a) obtaining a biological sample from the human;
(b) isolating DNA from the biological sample; and
(c) detecting the presence or absence of at least one allelic variant of a N-acetylglucosaminyl transferase component GPI-1 (GPI-1) gene in the DNA.

47. The method of claim 46, wherein at least one variant is a G to A transversion at position 2577 of a N-acetylglucosaminyl transferase component GPI-1 (GPI-1) gene.

48. A method of determining a response of a human to a cardiovascular drug, said method comprising the steps of:
(a) obtaining a biological sample from the human;
(b) isolating DNA from the biological sample; and
(c) detecting the presence or absence of at least one allelic variant of a cytochrome C oxidase subunit VIb (COX6B) gene in the DNA or at least one allelic variant of a N-acetylglucosaminyl transferase component GPI-1 (GPI-1) gene in the DNA.

49. The method of claim 46, wherein the detecting step is performed by an assay selected from the group consisting of allele specific hybridization, primer specific extension, oligonucleotide ligation, restriction enzyme site analysis, and single-stranded conformation polymorphism analysis.

50. The method of claim 48, wherein the detecting step is performed by an assay selected from the group consisting of allele specific hybridization, primer specific extension, oligonucleotide ligation, restriction enzyme site analysis, and single-stranded conformation polymorphism analysis.

51. A microarray comprising a nucleic acid having a sequence of a polymorphic region from a human N-acetylglucosaminyl transferase component GPI-1 (GPI-1) gene.

52. The microarray of claim 51, wherein the polymorphic region comprises a locus selected from the group consisting of position 2577 of the human N-acetylglucosaminyl transferase component GPI-1 (GPI-1) gene, position 2829 of the human GPI-1 gene, position 2519 of the human GPI-1 gene, position 2289 of the human GPI-1 gene, position 1938 of the human GPI-1 gene, position 1563 of the human GPI-1 gene, position 2656 of the human GPI-1 gene, and position 2664 of the human GPI-1 gene.

53. The microarray of claim 52, wherein the polymorphic region comprises position 2577 of the human N-acetylglucosaminyl transferase component GPI-1 (GPI-1) gene.

54. A kit comprising:
(a) at least one probe specific for a polymorphic region of a human gene selected from the group consisting of cytochrome C oxidase subunit VIb (COX6B); N-acetylglucosaminyl transferase component GPI-1 (GPI-1); cholesterol ester transfer protein, plasma (CETP); apolipoprotein A-IV (APO A4); apolipoprotein A-I (APO A1); apolipoprotein E (APO E); apolipoprotein B (APO B); apolipoprotein C-III (APO C3); a gene encoding lipoprotein lipase (LPL); ATP-binding cassette transporter (ABC 1); paraoxonase 1 (PON 1); paraoxonase 2 (PON 2); 5,10-methylenetetrahydrofolate r reductase (MTHFR); a gene encoding hepatic lipase, E-selectin, G protein beta 3 subunit and angiotensin II type 1 receptor gene; and
(b) instructions for use.
